(19) United States
(12) Patent Application Publication
(10) Pub. No.: US 2004/0148649 A1
(43) Pub. Date: Jul. 29, 2004

(54) Repressor-mediated selection strategies

(75) Inventors: Derek Lydiate, Saskatoon (CA); Abdelali Hannoufa, Saskatoon (CA); Nicholas Bate, Urbandale, IA (US); Dwayne Hegedus, Saskatoon (CA)

Correspondence Address: HOWREY SIMON ARNOLD & WHITE, LLP, BOX 34, 301 RAVENSWOOD AVE., MENLO PARK, CA, 94025, US

(73) Assignee: HER MAJESTY THE QUEEN IN RIGHT OF CANADA AS REPRESENTED BY THE MINISTER OF AGRICULTURE AND FOO

(21) Appl. No.: 10/678,490

(22) Filed: Oct. 3, 2003

Related U.S. Application Data
(60) Provisional application No. 60/416,369, filed on Oct. 3, 2002.

(51) Int. Cl. A01H 1/00; C12N 15/82; C12Q 1/68

(52) U.S. Cl. 800/278; 435/6

(57) ABSTRACT

The present invention provides plant selection strategies to identify and select plants cells, tissue or entire plants which comprise a coding region of interest. The plant selection strategy of the present invention generally involves i) transforming the plant, or portion thereof with a first nucleotide sequence comprising a first regulatory region in operative association with a first gene, and an operator sequence, the first gene encoding a tag protein; ii) screening for the transformed plant; iii) introducing a second nucleotide sequence into the transformed plant, or portion thereof to produce a dual transgenic plant, the second nucleotide sequence comprising a second regulatory region, in operative association with a second gene, and a third regulatory region in operative association with a third gene, the second gene comprising a coding region of interest, the third gene encoding a repressor capable of binding to the operator sequence thereby inhibiting expression of the first gene, and; iv) selecting for the dual transgenic plant by identifying plants, or portions thereof deficient in the tag protein, or an identifiable genotype or phenotype associated therewith. The first gene may be a conditionally lethal gene and the tag protein may be a conditionally lethal protein.

### CROSS-REFERENCE TO RELATED APPLICATIONS

This application claims the benefit of U.S. Provisional Application No. 60/416,369, filed Oct. 3, 2002.

### FIELD OF THE INVENTION

The present invention relates to the plant selection strategies. More specifically, the present invention relates to strategies to select for transgenic plant cells, tissue or plants that comprise a coding region of interest.

### BACKGROUND OF THE INVENTION

Transgenic plants are an integral component of agricultural biotechnology and are indispensable in the production of proteins of nutritional or pharmaceutical importance. They also provide an important vehicle for developing plants that exhibit desirable traits, for example, herbicide and insect resistance, and drought and cold tolerance.

Expressing transgenic proteins in plants offers many advantages over expressing transgenic proteins in other organisms such as bacteria. First, plants are higher eukaryotic organisms and thus have the same or similar intracellular machinery and mechanisms which govern protein folding, assembly and glycosylation as do mammalian systems. Further, unlike fermentation-based bacterial and mammalian cell systems, protein production in plants is not restricted by physical facilities. For example, agricultural scale production of recombinant proteins by plants is likely to be significantly greater than that produced by fermentation-based bacterial and mammalian cell systems. In addition, the costs of producing recombinant proteins in plants may be 10- to 50-fold lower than conventional bacterial bioreactor systems (Kusnadi et al. 1997). Also, plant systems produce pathogen free recombinant proteins. Further, the ability to produce biologically-active recombinant proteins in edible plant tissues or extracts allows low-cost oral delivery of proteins such as antigens as feed additives, and potentially eliminates the need for expensive down-stream purification processes of the protein.

Production of transgenic plants expressing a protein of interest requires transforming a plant, or portions thereof with a suitable vector comprising a gene that encodes a protein of interest. Transformation protocols are well known in the art. Following transformation, there exists a mixture of transformed and non-transformed plant cells. Transformed plant cells contain the vector carrying the coding region of interest, whereas untransformed plant cells do not contain the coding region of interest. The next step is usually to select transformed plants cells comprising the coding region of interest from the untransformed plant cells.

Selectable markers are genes required to tag or detect the insertion of desirable genes and are normally required for the process of plant transformation. Historically, selectable markers have been based on antibiotic or herbicide selection. This has raised concern that they could confer advantageous characteristics if transferred to weeds and be perpetuated in wild populations or be transferred to micro-organisms and contribute to the accumulation of antibiotic resistance genes. The construction of an ideal selectable marker would involve a gene activity that is benign and confers no advantage to plants or other organisms, thereby substantially decreasing the risk for genetic “pollution” through perpetuation in the environment.

The development of a suitable system to positively select for the introduction of foreign genes into a cell preferably employs two inseparable components; a compound that functions rapidly to eliminate non-transformed cells, and a mechanism to inactivate such a compound or to abrogate its action. The latter function is most often provided by enzymes that inactivate the selective compound by catalyzing the addition of adducts to the molecule (eg. acetyltransferases and phosphotransferases), by enzymes that break critical bonds in the molecule (hydrolases) or by binding proteins that recognize and sequester the compound.

A wide array of genes have been used as selectable markers for plant transformation and include: 1) classical antibiotic resistance, for example kanamycin (Koziel et al., 1984), hygromicin (Lin et al., 1996), phleomycin (Perez et al., 1989) and methotrexate resistance (Eichholtz et al., 1987) and 2) elements of basic metabolic pathways, such as purine salvage (Petolino et al., 2000), amino acid metabolism (Perl et al., 1992), carbohydrate biosynthesis (Sonnewald and Ebneth, 2000; Privalle et al., 2000) some of which have been developed as herbicide tolerance genes (eg. glyphosate, Ye et al., 2001).

There are references that disclose non-antibiotic selection strategies for transgenic plants. For example, WO 00/37660 discloses methods and genetic constructs to limit outcrossing and undesired gene flow in crop plants. The application describes the production of transgenic plants that comprise recombinant traits of interest linked to repressible genes. The lethal genes are blocked by the action of repressor molecules produced by the expression of repressor genes located at a different genetic locus. A drawback of the application is that the repressor must be expressed in order to have the coding region of interest expressed. Failure to express the repressor results in expression of the lethal gene and causes the death of the plant. In many transgenic plants, it may be desirable to express a coding region of interest in the absence of other proteins such as a repressor. The system disclosed above does not allow for such expression.

WO 00/37060 discloses genetic constructs for the production of transgenic plants which can be selectively removed from a growing site by application of a chemical agent or physiological stress. The application discloses the linkage of a target gene for a trait of interest to a conditionally lethal gene, which can be selectively expressed to cause plant death. A drawback of the application is that transformed plants containing the conditionally lethal gene and coding region of interest must be selected for under sublethal conditions. Selecting for transformed plants under sublethal conditions is more difficult and more prone to errors than is selecting for plants under lethal conditions.

WO 94/03619 discloses a recombinant plant genome that requires the presence of a chemical inducer for growth and development. The recombinant plant comprises a gene cascade including a first gene which is activated by external application of a chemical inducer and which controls expression of a gene product which affects expression of a second gene in the genome of the plant. Survival and development of the plant is dependant upon either expression or non-expression of the second gene. Application of the inducer selects whether or not the plant develops. A drawback of the application is that activation of the conditionally lethal gene is restricted to the application of a substance which triggers the lethal phenotype.

WO 96/04393 discloses the use of a repressed lethal gene to limit the growth and development of hybrid crops. Specifically, expression of a lethal gene is blocked by a genetic element that binds a repressor protein. The nucleotide sequence which binds the repressor protein comprises sequences recognized by a DNA recombinase enzyme such as the Cre enzyme. Plants containing the repressed lethal gene are crossed with plants containing the DNA recombinase gene. The recombinase function in the resulting hybrid plant removes the specific blocking sequence and activates expression of the lethal gene so that no other plant generations may be produced. A limitation of this application is that the genetic constructs disclosed cannot control outcrossing of germplasm.

Other negative selection schemes have exploited the ability of Agrobacterium tumefaciens, the causative agent of crown gall disease and the vector routinely used for plant transformation, to induce neoplastic growth of plant tissues upon infection (Fraley et al., 1986). This phenomenon results from a localized increase in the levels of two phytohormones, cytokinin and auxin, brought about by the actions of Agrobacterium Ti plasmid-encoded genes. Cytokinin levels are affected by expression of isopentyl transferase, the product of the ipt gene, which catalyzes the formation of isopentyl-adenosine-5-monophosphate, the first step in cytokinin biosynthesis. The dependency of shoot formation on the presence of cytokinin was used by Kunkel and coworkers (1999) to select for transgenic events by virtue of the fact that only those calli expressing the ipt gene developed shoots. When incorporated into a transposable element, the absence of aberrant phenotype associated with ipt expression serves as a scoreable marker to identify lines no longer possessing the transgene, for example, a selectable antibiotic marker (Ebinuma et al., 1997).

The auxin, indoleacetic acid (IAA), is normally synthesized from indole via endogenous biochemical pathways. The Agrobacterium Ti plasmid possesses genes encoding two enzymes capable of catalyzing the transformation of tryptophan into IAA. The first reaction requires the product of the iaaM gene, encoding tryptophan monooxygenase, which converts tryptophan into indole acetamide (IAM). The second reaction is carried out by the product of the iaaH gene, indole acetamide hydrolase, which converts IAM into IAA (Budar et al., 1986). Since neither the iaaH gene nor the intermediate IAM exist within plant cells, exposure of plants expressing iaaH to IAM, or its analogue alpha-naphthalene acetamide, leads to auxin formation and neoplastic growth. This system has been demonstrated to function effectively as a selectable marker in tissue culture (Depicker et al., 1988; Karlin-Neumann et al., 1991) and as a scoreable marker in field applications (Arnison et al., 2000).

Selective expression of the iaaM and iaaH genes can also lead to tissue-specific phenotypes. This has been used to develop a genetic containment system whereby iaaM expression is governed by a seed-specific promoter altered to contain DNA binding sites for a transcriptional repressor protein. When constructs encoding both the auxin biosynthetic enzymes and repressor protein are within the same seed progenitor cell(s), the aberrant phenotype is averted. Conversely, if the two components become separated, such as through normal chromosome sorting during outcrossing, repression of auxin biosynthesis in relieved leading to seed lethality (Fabijanski et al., 1999). If a particular transgene is physically linked to the auxin biosynthetic genes it will also be prevented from propagating outside of the original plants genetic context.

In many instances, the expression of transgenes needs to be repressed in certain plant organs/tissues or at certain stages of development. Gene repression can be used in applications such as metabolic engineering and producing plants that accumulate large amounts of certain compounds. Repression of gene expression can also be used for control of transgenes across generations, or production of F1 hybrid plants with seed characteristics that would be undesirable in the parents, i.e. hyper-high oil. An ideal repression system should exhibit some level of flexibility, and avoid external intervention or subjecting the plant to various forms of stress. Such a system should also combine at least the following four features:

1. The repressor should not be toxic to the plant and its ecosystem.

2. Repression should be restricted to the target gene.

3. The target gene should have normal expression levels in the absence of the repressor.

4. In the presence of the repressor, the expression of the target gene should be undetectable.

A small number of prokaryotic gene repressors, e.g. TetR (Gatz et al., 1992) and LacR (Moore et al., 1998), have been engineered to be used for gene regulation in plants. Repression of gene expression can be accomplished by introducing operator sequences specific for the binding of known repressors, e.g. TetR and LacR, in the promoter region of desirable genes in plants expressing the repressor. Some repressors, such the E. coli LacI gene product, LacR, function by blocking transcription initiation as well as transcript elongation. Insertion of Lac operators in the promoter region results in blocking transcription initiation (Bourgeois and Pfahl, 1976), whereas placing them in the transcribed region led to the premature termination of the transcript (Deuschle et al., 1990). The action of TetR, on the other hand, appears to be restricted to preventing transcript initiation. Placing Tet operators in the upstream untranslated region of the CaMV35S was not effective in repressing transcription, whereas inserting them in the vicinity of the TATA box resulted in blocking transcript initiation (Gatz and Quayle, 1988; Gatz et al., 1991). A stringent Tet repression system was constructed using the CaMV35S promoter by placing one Tet operator immediately upstream of the TATA box and two downstream of the TATA box, but upstream of the transcription initiation site (Gatz et al., 1992). However, this system was found to be inoperable in many plant species, including Brassica napus and Arabidopsis thaliana.

There is a need in the art for selectable marker systems for plant transformation that are not based on antibiotic resistance. Further there is a need in the art for a selectable marker system for plant transformation that is benign to the transformed plant and confers no advantage to other organisms in the event of gene transfer. There is also a need for a simple method of selection. Further, there is a need in the art for a selectable marker system for plant transformation that includes stringent selection of transformed cells, avoids medically relevant antibiotic resistance genes, and uses an inexpensive and effective selection agent that is non-toxic to plant cells.

It is an object of the invention to provide a plant select strategy.

### SUMMARY OF THE INVENTION

The present invention relates to the repressor-mediated selection strategies. More specifically, the present invention relates to strategies to select for transgenic plant cells, tissue or plants that comprise a coding region of interest.

The present invention provides a method of selecting for a plant or portion thereof that comprises a coding region of interest, the method comprising,

i) providing a platform plant, or portion thereof comprising a first nucleotide sequence comprising,

a first regulatory region in operative association with a first coding region, and an operator sequence, the first coding region encoding a tag protein;

ii) introducing a second nucleotide sequence into the platform plant, or portion thereof to produce a dual transgenic plant, the second nucleotide sequence comprising,

a second regulatory region, in operative association with a second coding region, and a third regulatory region in operative association with a third coding region, the second coding region comprising a coding region of interest, the third coding region encoding a repressor capable of binding to the operator sequence thereby inhibiting expression of the first coding region, and;

iv) selecting for the dual transgenic plant by identifying plants, or portions thereof deficient in the tag protein, expression of the first coding region, or an identifiable genotype or phenotype of the dual transgenic plant associated therewith.

The present invention also pertains to a method of selecting for a plant or portion thereof that comprises a coding region of interest, the method comprising,

i) transforming the plant, or portion thereof with a first nucleotide sequence comprising,

a first regulatory region in operative association with a first coding region, and an operator sequence, the first coding region encoding a tag protein;

ii) introducing a second nucleotide sequence into the transformed plant, or portion thereof to produce a dual transgenic plant, the second nucleotide sequence comprising,

a second regulatory region, in operative association with a second coding region, and a third regulatory region in operative association with a third coding region, the second coding region comprising a coding region of interest, the third coding region encoding a repressor capable of binding to the operator sequence thereby inhibiting expression of the first coding region, and;

iii) selecting for the dual transgenic plant by identifying plants, or portions thereof deficient in the tag protein, the first coding region, or an identifiable genotype or phenotype associated therewith.

The plant or portion thereof may comprise plant cells, tissue or one or more entire plants. Further, the plant or portion thereof may be selected from the group consisting of canola, Brassica spp., maize, tobacco, alfalfa, rice, soybean, pea, wheat, barley, sunflower, potato, tomato, and cotton. The first coding region is selected from the group consisting of a reporter protein, an enzyme, an antibody and a conditionally lethal coding region.

Also according to the method of the present invention as defined above, the conditionally lethal coding region may be any conditionally lethal coding region known in the art. Preferably, the conditionally lethal coding region is selected from the group consisting of indole acetamide hydrolase, methoxinine dehydrogenase, rhizobitoxine synthase, and L-N-acetyl-phosphinothricin deacylase. In an aspect of an embodiment, the conditionally lethal coding region is indole acetamide hydrolase.

Further according to the method of the present invention as defined above, the repressor and the operator sequence may be selected from the group consisting of

a) Ros repressor and Ros operator sequence;

b) Tet repressor and Tet operator sequence;

c) Sin3 repressor and Sin3 operator sequence; and

d) UTMe6 repressor and UTMe6 operator sequence.

Preferably, the repressor and operator sequence is the Ros repressor and Ros operator sequence or the Tet repressor and Tet operator sequence.

Also according to the method of the present invention as defined above, the coding region of interest may encode a pharmaceutically active protein such as, but not limited to, growth factors, growth regulators, antibodies, antigens, interleukins, insulin, G-CSF, GM-CSF, hPG-CSF, M-CSF, interferons, blood clotting factors, transcriptional protein or nutraceutical proteins.

Further, according to an aspect of an embodiment of the present invention according, there is provided a method of selecting for a transgenic plant or portion thereof comprising a coding region of interest, the method comprising,

i) transforming the plant, or portion thereof, with a first nucleotide sequence to produce a transformed plant, the first nucleotide sequence comprising a first regulatory region in operative association with a first coding region, and an operator sequence, the first coding region encoding a conditionally lethal protein;

ii) screening for the transformed plant;

iii) introducing a second nucleotide sequence into the transformed plant or portion thereof to produce a dual transgenic plant, the second nucleotide sequence comprising a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region, and;

iv) selecting for the dual transgenic plant by exposing the transformed plant and the dual plant to conditions that permit the conditionally lethal coding region to become conditionally lethal, thereby reducing the growth, development or killing the transformed plant.

The plant, or portion thereof may comprise plant cells, tissue or entire plant.

Also according to the method of the present invention as defined above the first regulatory region, secondary regulatory region and third regulatory region may be constitutively active in the plant cells. Alternatively, but not to be limiting in any manner, the first regulatory region and secondary regulatory region may be constitutively active and the third regulatory region may be developmentally regulated or inducible.

Also, according to an aspect of an embodiment of the present invention, there is provided a method of selecting for a transgenic plant or portion thereof comprising a coding region of interest, the method comprising,

i) introducing a second nucleotide sequence into a transformed plant, or portion thereof that comprises a first nucleotide sequence to produce a dual transgenic plant, the first nucleotide sequence comprising a first regulatory region in operative association with a first coding region, and an operator sequence, the first coding region encoding a conditionally lethal protein,

and wherein said second nucleotide sequence comprises a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region, and;

ii) selecting for the dual transgenic plant by exposing the transformed plant and the dual transgenic plant to conditions that permit the conditionally lethal coding region to become conditionally lethal, thereby reducing the growth, development or killing the transformed plant.

Further, according to an aspect of an embodiment of the present invention, there is provided a method of selecting for a transgenic plant or portion thereof comprising a coding region of interest, the method comprising,

i) transforming the plant, or portion thereof, with a first nucleotide sequence to produce a transformed plant, the first nucleotide sequence comprising a first regulatory region in operative association with a first coding region, and an operator sequence, the first coding region encoding a conditionally lethal protein;

ii) screening for the transformed plant;

iii) introducing a second nucleotide sequence into the transformed plant or portion thereof to produce a dual transgenic plant, a second nucleotide sequence comprising a second regulatory region in operative association with a second coding region encoding a fusion-protein, the fusion protein comprising a protein of interest fused to a repressor capable of binding to the operator sequence of the first coding region thereby inhibiting expression of the first coding region, and;

iv) selecting for the dual transgenic plant by exposing the transformed plant and the dual transgenic plant to conditions that permit the conditionally lethal coding region to become conditionally lethal, thereby reducing the growth, development or killing the transformed plant, or portion thereof.

Further, the fusion-protein as defined above may comprise a linker region linking the repressor to the protein of interest, an affinity tag, or both. The linker region may be enzymatically cleavable to separate the protein of interest from the repressor. Preferably the fusion-protein has a molecular mass less than about 100 kDa, more preferably less than about 65 kDa or comprises a sequence.

Also according to an aspect of an embodiment of the present invention, there is provided a plant cell, tissue, seed or plant comprising,

i) a first nucleotide sequence comprising a first regulatory region in operative association with a first coding region, said first coding region encoding a tag protein, and;

ii) a second nucleotide sequence comprising a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region.

The first coding region may comprise, but is not limited to a conditionally lethal coding region and the tag protein may comprise but is not limited to a conditionally lethal protein.

Also, according to an aspect of an embodiment of the present invention there is provided a plant cell, tissue, seed or plant comprising,

i) a first nucleotide sequence comprising a first regulatory region in operative association with a first coding region, said first coding region encoding a tag protein, and;

ii) a second nucleotide sequence comprising a second regulatory region in operative association with a second coding region, the second coding region encoding a fusion-protein, said fusion-protein comprising a protein of interest fused to a repressor capable of binding to the operator sequence thereby inhibiting expression of the first coding region.

The present invention also provides a plant cell, tissue, seed or plant comprising, a first nucleotide sequence comprising a first regulatory region in operative association with a first coding region and an operator sequence, the first coding region encoding a tag protein.

The present invention also is directed to providing a plant cell, tissue, seed or plant comprising, a second nucleotide sequence comprising a second regulatory region in operative association with a second coding region, and a third regulatory region in operative association with a third coding region, the second coding region comprising a coding region of interest, the third coding region encoding a repressor capable of binding to an operator sequence.

Furthermore, the present invention is directed to a construct comprising, a first nucleotide sequence comprising a first regulatory region in operative association with a first coding region and an operator sequence, the first coding region encoding a tag protein.

The present invention pertains to a construct comprising a second nucleotide sequence comprising a second regulatory region in operative association with a second coding region, and a third regulatory region in operative association with a third coding region, the second coding region comprising a coding region of interest, the third coding region encoding a repressor capable of binding to an operator sequence.

The present invention also provides a pair of constructs comprising,

i) a first nucleotide sequence comprising a first regulatory region in operative association with a first coding region and an operator sequence, the first coding region encoding a tag protein, and;

ii) a second nucleotide sequence comprising a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region.

Alternatively, the present invention pertains to a pair of constructs comprising,

i) a first nucleotide sequence comprising a first regulatory region in operative association with a first coding region and an operator sequence, the first coding region encoding a tag protein, and;

ii) a second nucleotide sequence comprising a second regulatory region in operative association with a second coding region, the second coding region encoding a fusion-protein, the fusion-protein comprising a protein of interest fused to a repressor capable of binding to the operator sequence thereby inhibiting expression of the first coding region.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a diagrammatic representation of the conversion of tryptophan to indole-3-acetamide (IAM) by IAAM (tms1) and the subsequent conversion of indole-3-acetamide (LAM) to Indole-3-acetic acid (IAA) by IAAH (tms2).

FIG. 2 shows a non-limiting example of genetic constructs described by the present invention, wherein expression of a coding region of interest and coding region encoding the repressor protein are controlled by separate regulatory sequences.

FIG. 3 shows several alternate non-limiting examples of genetic constructs described by the present invention, wherein expression of a coding region of interest and coding region encoding the repressor protein are controlled by the same regulatory sequence.

FIG. 4 shows nucleotide sequences for the Ros operator sequence and Ros repressor. FIG. 4A shows the nucleotide sequence of the operator sequences of the virC/virD (SEQ ID NO: 17) and ipt genes (SEQ ID NO:18). FIG. 4B shows a consensus operator sequence (SEQ ID NO:23) derived from the virC/virD (SEQ ID NO:57-58) and ipt (SEQ ID NO: 59-60) operator sequences shown in FIG. 4A. The consensus sequence comprises 10 nucleotides, however, only the first 9 nucleotides are required for binding ROS. FIG. 4C shows a Ros sequence derived from Agrobacterium tumefaciens (upper strand; SEQ ID NO: 19) and a synthetic Ros sequence optimized for plant expression (lower strand; SEQ ID NO: 1). Nucleotides that are shaded indicate identical nucleotides. FIG. 4D shows Southern analysis of a plant comprising a first nucelotide sequence, p74-309 (35S with two ROS operator sequences operatively linked to GUS; see FIG. 9C for map). FIG. 4E shows Southern analysis of a plant comprising a second nucelotide sequence, p74-101 (actin2-synthetic ROS; see FIG. 9A for map). FIG. 4F shows Western analysis of ROS expression in transformed Arabidopsis plants. Levels of wild type ROS, p74-107 (35S-WTROS; see FIG. 11 for map), and synthetic ROS p74-101 (actin2-synROS; see FIG. 9A for map) produced in transgenic plants were determined by Western analysis using a ROS polyclonal antibody. Arabidopsis var. columbia, was run as a control. FIG. 4G shows expression of a first nucleotide sequence (10, FIG. 2) in plants. Upper panel shows expression of GUS under control of a 35S promoter(pBI121; comprising 35S-GUS). Middle panel shows GUS expression under control of actin2 promoter comprising a Ros operator sequence (p74-501; see FIG. 9A, Table 3 Examples for construct). Lower panel shows the lack of GUS activity in a non-transformed control.

FIG. 5 shows a Tet nucleotide sequence derived from E. coli tn10 transposon (Accession No. J01830; upper strand; SEQ ID NO:20) and a synthetic Tet sequence optimized for plant expression (lower strand; SEQ ID NO:2). Nucleotides that are shaded indicate identical nucleotides.

FIG. 6 shows the protein coding region of wild-type Ros (lower strand; SEQ ID NO:21) and synthetic Ros sequence (upper strand; SEQ ID NO:3). The protein coding region of the nucleotide sequence of the synthetic Ros sequence, and comprises the nuclear localization signal “PKKKRKV” (SEQ ID NO:24).

FIG. 7 shows the protein coding region of wild-type Tet (lower strand; SEQ ID NO:22)and synthetic Tet sequence (upper strand; SEQ ID NO:4) wherein the protein coding region of the nucleotide sequence was optimized for expression in plants, and comprises the nuclear localization signal “PKKKRKV” (SEQ ID NO:24).

FIG. 8 shows results of Northern blot analysis on 74-502 (85, 170 and 176) and 74-503 (86, 82 and 83) plant lines. Wt is wildtype. Probes for Northern analysis were generated with radiolabelled tms2 ORF EcoRV/BglII fragment

FIG. 9 shows maps of several non-limiting constructs used in the present invention FIG. 9A shows p74-101 (actin2-synRos), p74-313 (35S-synRos), p74-316 (35S-RosOS-GUS); p74-118 (35S-3x RosOS-GUS), p74-117 (35S-3x RosOS-GUS), p74-501 (actin2-RosOS-GUS). FIG. 9B shows p74-315 (35S-RosOS-GUS). FIG. 9C shows p74-309 (35S-2x RosOS-GUS). FIG. 9D shows p76-508 (tms2-2x RosOS-GUS). FIG. 9E shows p74-107 (35S-Ros). FIG. 9F shows p74-108 (tms2-synRos).

FIG. 10 shows results of Western Blot analysis of Ros and Tet repressors expressed in transgenic Arabidopsis thaliana lines. FIG. 10A shows transgenic plant lines expressing synthetic Ros repressor under the control of actin2 (RS-318,19,25,26,29, 30) or iaaH (RS-69) promoters. FIG. 10B shows transgenic plant lines p75-103 expressing synthetic Tet repressor under the control of actin2 promoter. Anti-Tet antibody was used as a probe.

FIG. 11 shows non-limiting examples of several constructs of the present invention.

FIG. 12 shows results of plant selection using the method of the present invention. FIG. 12A shows results of GUS assays of two parent plants, one expressing the first nucleotide sequence comprising GUS as a tag protein (GUS parent), the other comprising the second nucleotide sequence and expressing Ros as the third coding region (ROS parent), and of a progeny of a cross between the GUS and ROS parents (cross). FIG. 12B shows results of Northern analysis using either a GUS probe or a Ros probe, of two parent plants, GUS parent and ROS parent, and a progeny of a cross between the GUS and Ros parents (cross). FIG. 12C shows a Southern analysis using either a GUS probe or a Ros probe, of the GUS parent and ROS parent plants.

FIG. 13 shows Northern analysis of tag protein expression from a series of parental lines and progeny from crosses of parental lines expressing tag protein and parental lines expressing repressor protein. Total RNA (˜4.5 g) was isolated from Arabidopsis parental lines expressing tag protein, in this case GUS and crosses between various combinations of parental lines expressing GUS and Ros (C1-C5; see FIG. 9A for constructs; see Table 6, Example 5 for crosses). Parental transgenic plants and progeny arising from the crosses were analyzed for GUS using a GUS probe (FIG. 13A). FIG. 13A also shows loading of the RNA gel. FIG. 13B shows quantification of the densities of bands generated by Northern blot analysis of total RNA isolated from Arabidopsis reporter-repressor crosses and parental lines and probed with GUS (FIG. 13A). Plant lines are as indicated in Example 5. Band intensity was calculated using Quantity One Software (Biorad).

FIG. 14 shows nuclear localization of GUS, wtRos-GUS, and synRos-GUS proteins in onion cells. FIG. 14A is a schematic diagram of (GUS), p74-132 (wtRos-GUS) and p74-133 (synRos-GUS) constructs. The synRos and wtRos ORFs were fused in-frame to the GUS reporter gene and driven by the CaMV35S. FIG. 14B shows transient expression of GUS, wtRos-GUS and synRos-GUS proteins in onion cells. Onion tissues were analyzed using histochemical GUS assay (left) and nucleus-specific staining with DAPI (right).

FIG. 15 shows binding of the synRos protein to the Ros operator. Double stranded Ros operator (1); single stranded Ros operators in sense (2) and antisense (3) orientations respectively; negative control single stranded oligonucleotides from the TetR operator sequence in the sense (4) and antisense (5) orientations.

FIG. 16 shows GUS expression under the modified and unmodified CaMV35S promoters. FIG. 16A shows GUS expression in Arabidopsis control crosses under the unmodified CaMV35S promoter (pBI121). The top panel shows a Northern blot analysis of RNA from Arabidopsis plants, probed with GUS. Lines are crosses between plants expressing p74-101 construct and plants expressing pBI121, or parental GUS and Ros plants. The bottom panel shows a EtBr stained RNA gel showing equal loading. FIG. 16B shows GUS expression in Arabidopsis under the modified CaMV35S promoters. The top panel shows a Northern blot analysis of RNA from Arabidopsis plants transformed with p74-117, p74-118 or pBI121 contructs. The bottom panel show a EtBr stained RNA gel to show equal loading.

FIG. 17 shows Northern blot analysis of total RNA isolated from Brassica napus reporter/repressor crosses and parental lines. In FIGS. 13A-B transgenic B. napus plants were crossed and analyzed for expression level of the GUS gene. The female parent is indicated first. Crosses performed are as follows: C1 to C4 are p74-114 x p74-101. P1 to P4 are GUS parent lines for crosses C1 to C4. FIG. 17A shows a Northern blot analysis of B. napus GUS x Ros crosses and GUS parental lines. Ethidium bromid stained total RNA is also shown to indicate RNA loading. FIG. 17B shows quantification of the Repression levels. Relative values of the densities of bands generated by Northern blot analysis were expressed as a percentage of the densities of the repective 28s rRNA bands on the gel.

### DETAILED DESCRIPTION

The present invention relates to the repressor-mediated selection strategies. More specifically, the present invention relates to strategies to select for transgenic plant cells, tissue or plants that comprise a coding region of interest.

The following description is of a preferred embodiment.

According to an aspect of the present invention, there is provided a method of selecting for a plant that comprises a coding region of interest. The method comprises,

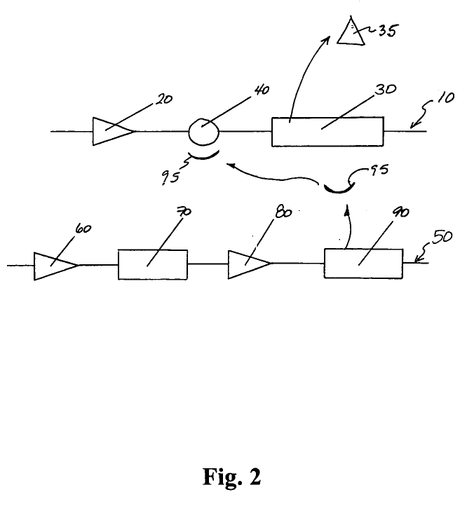

i) transforming the plant, or portion thereof with a first nucleotide sequence (10; FIG. 2) to produce a transformed plant, the first nucleotide sequence (10) comprising, a first regulatory region (20) in operative association with a first coding region (30), and an operator sequence (40), the first coding region encoding a tag protein (35);

ii) introducing a second nucleotide sequence (50) into the transformed plant, or portion thereof to produce a dual transgenic plant, the second nucleotide sequence comprising, a second regulatory region (60) in operative association with a second coding region (70), and a third regulatory region (80) in operative association with a third coding region (90), the second coding region (70) comprising a coding region of interest, the third coding region (90) encoding a repressor (95) capable of binding to the operator sequence (40) thereby inhibiting expression of the first coding region (30);

iii) selecting for the dual transgenic plant by identifying plants deficient in the tag protein (35), or an identifiable genotype or phenotype associated therewith.

The method may also include a step of screening for a transformed plant, expressing the tag protein, prior to the step of introducing (step ii)).

The step of introducing (step ii)) may comprise any step as known in the art, for example but not limited to, transformation or cross breeding.

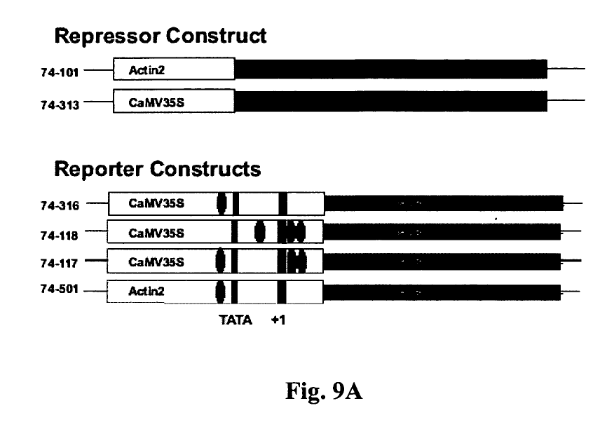

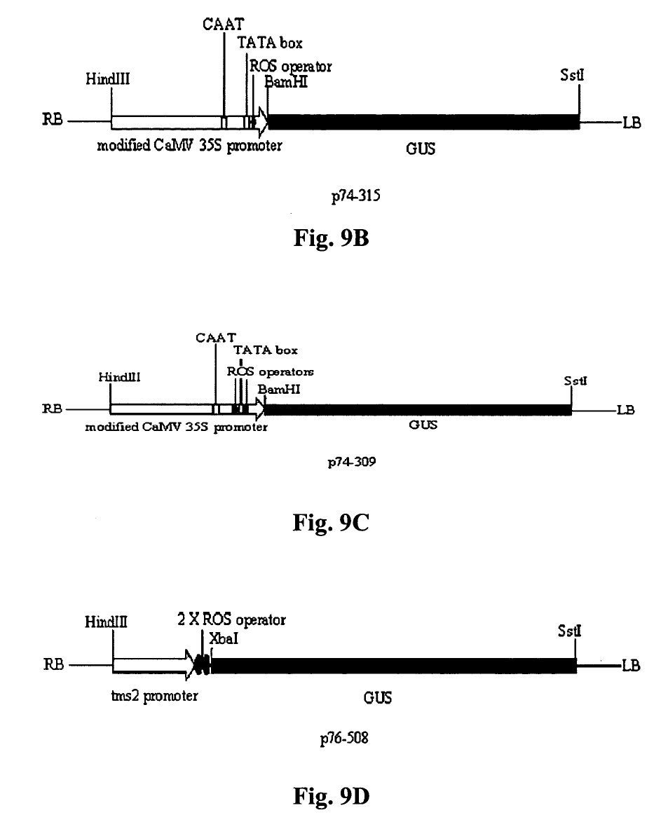

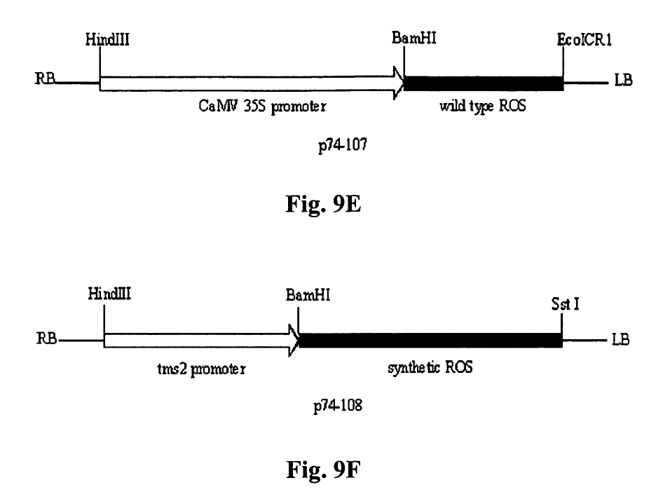

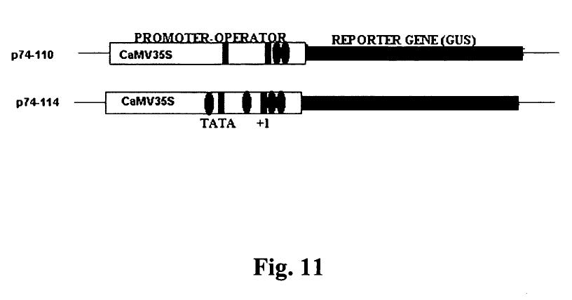

By the term “tag protein” it is meant any protein that is capable of being identified in a plant. For example, but not wishing to be limiting, the tag protein may be an enzyme that catalyzes a reaction, for example GUS. In such an embodiment the enzyme may be identified by an enzymatic assay. Alternatively, but without wishing to be limiting, the tag protein may be an immunogen and identified by an immunoassay, or the tag protein may confer an observable phenotype, such as, but not limited to the production of green fluorescent protein (GFP). Other methods for the detection of the expression of the first coding region (30) may be used, including but not limited to, Northern hybridization, S1 nuclease, array analysis, PCR, or other methods as would be known to one of skill in the art. The tag protein may also be a positive selection marker, for example, a conditionally lethal protein which is encoded by a conditionally lethal sequence (the first coding region), resulting in an observable phenotype, for example wilting or death of a plant or a portion thereof. Non-limiting examples of constructs comprising a first coding region (30) encoding a tag protein (35) include constructs listed in Table 3 (see Examples) and in FIG. 9A (p74-316; p74-118; p74-117; p74-501), FIG. 9B (p74-315), FIG. 9C (p74-309), FIG. 9D (p74-508), and FIG. 11 (p74-110, p74-114).

By the term “conditionally lethal sequence” or “conditionally lethal protein”, it is meant a nucleotide sequence which encodes a protein, or the protein encoded by the conditionally lethal sequence, respectively, that is capable of converting a substrate to a product that alters the growth or development of a plant or a portion thereof, or that is capable of converting a substrate to a product that is a toxic to the plant, or portion thereof. The substrate is preferably a non-toxic substrate that may be produced by the plant or a portion thereof, or the substrate may be exogenously applied to the plant or portion thereof. Non-limiting examples of constructs comprising a conditionally lethal sequence encoding a conditionally lethal protein (tag protein) include p74-311, p74-503, p76-509, and p76-510 (Table 4 see Examples).

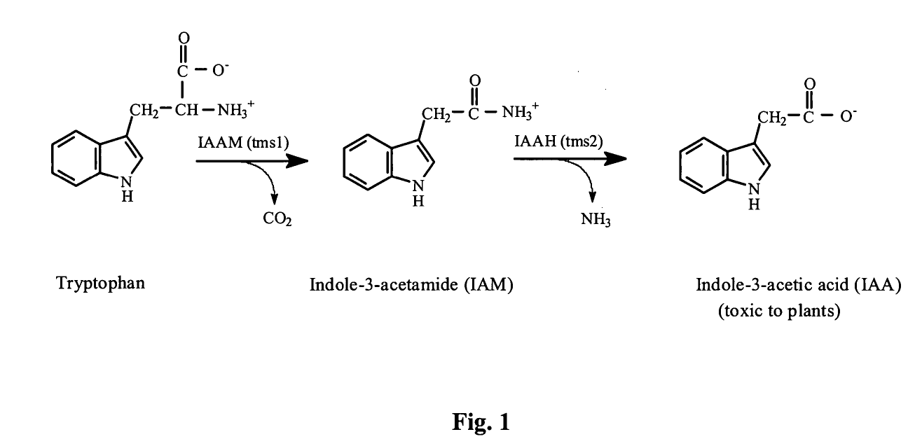

By the term “non-toxic substrate” it is meant a chemical substance that does not substantially affect the metabolic processes, or the growth and development of a plant or a portion thereof. A non toxic substrate may be endogenous within the plant or portion thereof, for example but not limited to indole acetamide (LAM; see FIG. 1) at concentrations typically found within a plant, or it may be applied to the plant or portion thereof, for example but not limited to indole napthal-3-acetamide (NAM; also referred to as naphalene acetamide)

The term “toxic product” or “a product that is toxic”, refers to a chemical substance which substantially affects one or more metabolic processes of a plant cell, tissue, or whole plant. A toxic product may impair growth, development, or impair both growth and development of a plant or portion thereof. Alternatively, a toxic product may kill the plant, or portion thereof. Preferably, the effect of the toxic product is detected by visual inspection of the plant or portion thereof, allowing for a ready determination of the expression of the first coding region (30), encoding the tag protein (35). However, other methods for the detection of the expression product of the first coding region (30) may also be used, including but not limited to, Northern hybridization, S1 nuclease, array analysis, PCR, or other methods as would be known to one of skill in the art.

Any conditionally lethal sequence known in the art that is capable of encoding a protein that converts a non-toxic substrate to a toxic product may be used in the method of the present invention provided that the toxic product is capable of altering the growth and development of the plant or portion thereof. Examples of a tag protein that is a conditionally lethal proteins, and which is not to be considered limiting in any manner, includes indole acetamide hydrolase (IAAH; tms2, FIG. 1), methoxinine dehydrogenase, rhizobitoxine synthase, or L-N-acetyl-phosphinothricin deacylase (PD), and enzymes involved in herbicide resistance, for example but not limited to ESPS synthase or phosphonate monoester hydrolase (U.S. Pat. No. 5,180,873; Margraff et al.,1980; Owens et al., 1973; EP 617121; CA 1,313,830; U.S. Pat. No. 5,254,801 and which are herein incorporated by reference):

IAAH (tms2) converts the non-toxic substrates indole acetamide (IAM), or indole napthalacetimide (NAM), to indole acetic acid (IAA; FIG. 1), or indole napthal acetic acid (NAA), respectively. The products, LAA or NAA, are toxic at elevated concentrations within a plant or portion thereof (U.S. Pat. No. 5,180,873);

methoxinine dehydrogenase converts the non-toxic substrate 2-amino4-methoxybutanoic acid (methoxinine) to the toxic product methoxyvinyl glycine (R. Margraff et al., 1980);

rhizobitoxine synthase converts the non-toxic substrate 2-amino-4-methoxybutanoic acid to the toxic product 2-amino-4-[2-amino-3-hydroxypropyl]-trans-3-butanoic acid (rhizobitoxine);

L-N-acetyl-phosphinothricin deacylase (PD) converts the non-toxic substrate N-acetyl-phosphinothricin to the toxic product phosphinothricin (L. D. Owens et al., 1973);

an enzyme that confers herbicide resistance, for example, EPSP synthase (CA 1,313830) or phosphonate monoester hydrolase which metabolizes glyphosate (U.S. Pat. No. 5,245,801).

Conditions that permit the conditionally lethal protein to become conditionally lethal, thereby reducing the growth, development, or killing, the transformed plant, include:

activation of the first regulatory region (20) which is in operative association with the first coding region (30) encoding a conditionally lethal protein (tag protein; 35). Ectopic expression of the conditionally lethal protein (tag protein) results in the utilization of an endogenous substrate (for example but not limited to IAM) to produce a product (e.g. IAA) that at elevated concentrations reduces growth, development, or kills the plant. The first regulatory region (20) may be developmentally regulated, tissue specific or an inducible regulatory region;

applying a non-toxic substrate to a plant expressing the tag protein (35) so that the non-toxic substrate is converted to a product that is toxic. The first regulatory region (20) may be any suitable regulatory region including, constitutively expressed, developmentally regulated, tissue specific, or an inducible regulatory region.

As will be evident to someone of skill in the art, the term “non-toxic” and “toxic” are relative terms and may depend on factors such as, but not limited to the amount of the substrate, the growth conditions of the plant or portion thereof, and if exogenously applied, the conditions under which the substrate is applied. If the non-toxic substrate is applied to the plant or portion thereof, the substrate is applied at a dose which has little or no adverse effect on the plant or a portion thereof, in the absence of the tag protein. The non-toxic substrate is converted to a product that is toxic if the tag protein (35), in this case encoded by the conditionally lethal sequence (20) is expressed by the plant or a portion thereof. The appropriate amount of non-toxic substrate to be applied to a plant may be readily determined. For example, which is not to be considered limiting if the non-toxic substrate is NAA, then from about 1 μM to about 5 μM NAA may be applied to a plant or a portion thereof, that expresses IAAH (a tag protein), resulting in a visual marker for the expression of the conditionally lethal sequence.

By the term “selecting” it is meant differentiating between a plant or a portion thereof, that:

i) expresses the first coding region (30) encoding the tag protein (35), from a plant that does not express the tag protein, or that

ii) expresses the second nucleotide sequence (50) including the coding region of interest (the second nucleotide sequence; 70) and the third coding region (90) encoding the repressor (95), from a plant, or portion thereof, which lacks the coding region of interest (70), for example in a dual transgenic plant.

Selecting may involve, but is not limited to, detecting the presence of the tag protein (35), activity associated with the tag protein (35), or expression of the first coding region (30) using standard methods. If the tag protein is a marker such as a GFP, then the presence of GFP may be detected using standard methods, for example using UV light. If the tag protein is an enzyme or an antigen, this activity can be assayed, for example assaying for GUS activity, or an ELISA or other suitable test, respectively. Similarly, the expression of the first nucleic acid sequence may be determine by assaying for the transcript, for example but not limited to, using Northern hybridization, S1 nuclease, array analysis, PCR, or other methods as would be known to one of skill in the art. If the tag protein is a conditionally lethal sequence, then in the presence of a toxic substrate, alteration in the growth, the development, or killing, of the plant or portion thereof, occurs and identifies plants that express the first coding region (30) encoding the tag protein (35; in this case a conditional lethal protein). In this way selecting may be used to differentiate between a plant which lacks the second nucleotide sequence (50) comprising the coding region of interest (70), and the third gene that encodes the repressor (90) from a plant that expresses the second nucleotide sequence (50), since if the repressor is present, then the repressor binds the operator sequence (40) of the first nucleotide sequence (10), and inhibits or reduces expression of the first coding region (30), and tag protein levels are reduced. Conversely, if the tag protein is present, then visual inspection of the plant or portion thereof indicates either that the first nucleotide construct has been introduced into the plant, as in i) above, or that the plant or portion thereof has not been transformed with the second nucleotide sequence, as in ii) above.

The term “plant, or portion thereof” refers to a whole plant, or a plant cell, including protoplasts or other cultured cell including callus tissue, or parts of a plant, including organs, for example but not limited to a root, stem, leaf, flower, anther, pollen, stamen, pistil, embryo, seed, or other tissue obtained from the plant.

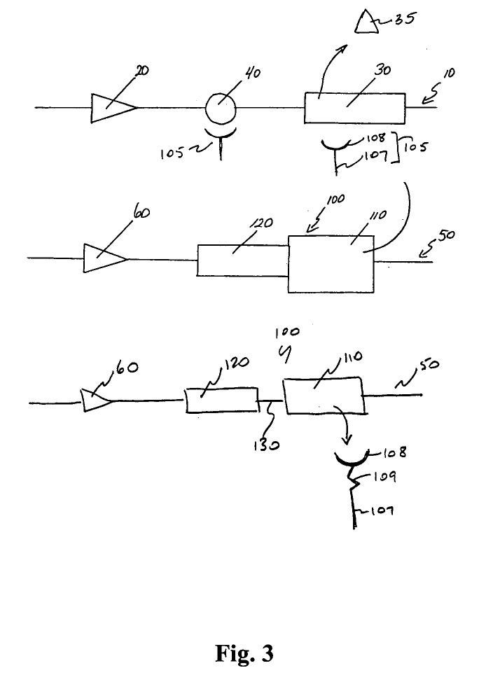

By the term “operator sequence” it is meant a nucleotide sequence which is capable of binding with a repressor, a peptide or a fusion protein, provided that the repressor, peptide or fusion protein comprise an appropriate operator binding domain. The operator sequence (40) is preferably located in proximity of a first coding region (20), either upstream, downstream, or within, the coding region, for example within an intron. When a repressor protein (95), or the DNA binding domain (108, FIG. 3) of the repressor, binds the operator sequence (40) expression of the coding region (30) that is in operative association with the operator sequence is reduced or inhibited. Preferably, the operator sequence is located in the proximity of a regulatory region (20) that is in operative association with the first coding region (30). However, the operator sequence may also be localized elsewhere within the first nucleotide sequence (10) to block migration of polymerase along the nucleic acid.

An operator sequence may be a Tet operator sequence (U.S. Pat. No. 6,117,680; U.S. Pat. No. 6,136,954; U.S. Pat. No. 5,646,758; U.S. Pat. No. 5,650,298; U.S. Pat. No. 5,589,362 which are incorporated herein by reference), a Ros operator sequence, or a nucleotide sequence known to interact with a DNA binding domain of a protein. In this latter case, it is preferred that the protein comprising the DNA binding domain is fused to a repressor. Non-limiting examples of DNA binding domains that may be used, where the DNA binding domain counterpart is fused to a repressor, include Gal4, Lex A, ZFHD1 domain, hormone receptors, for example steroid, progesterone or ecdysone receptors and the like.

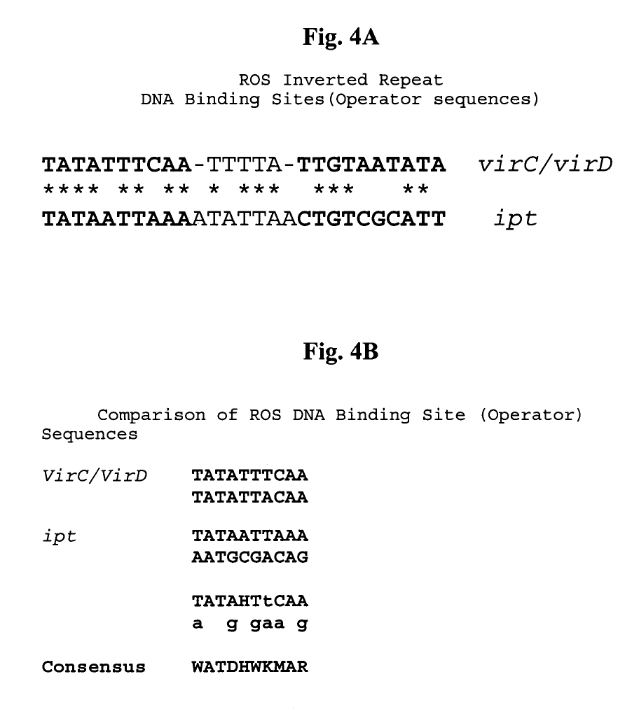

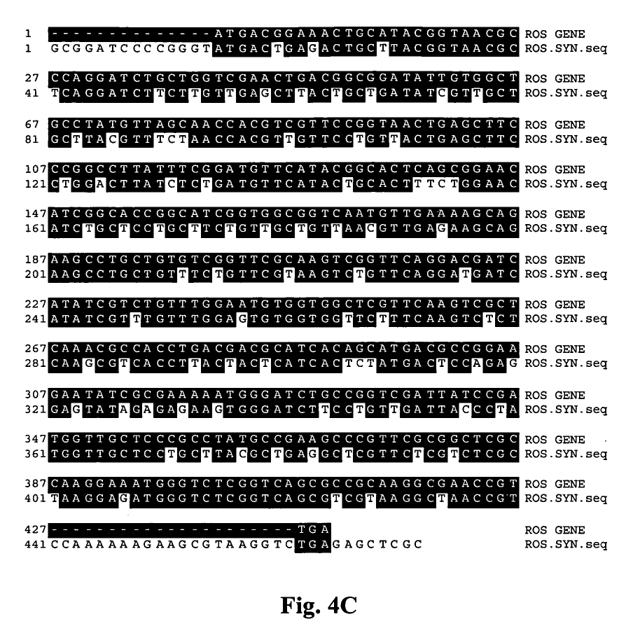

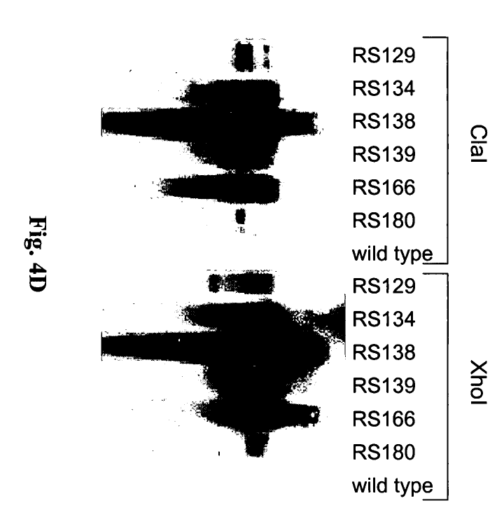

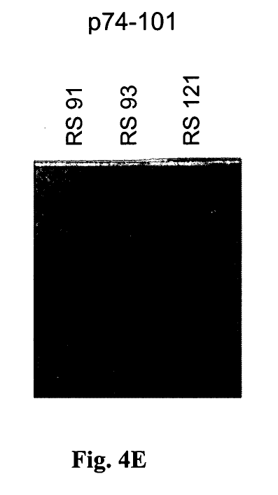

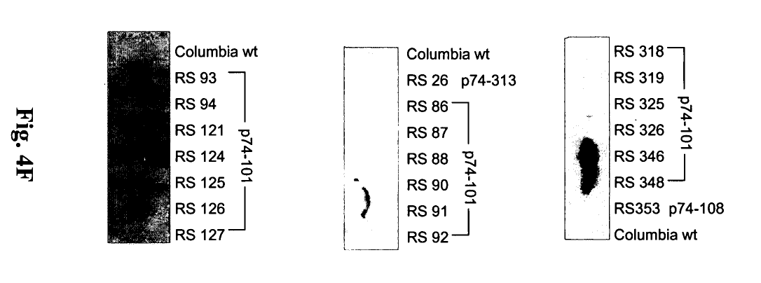

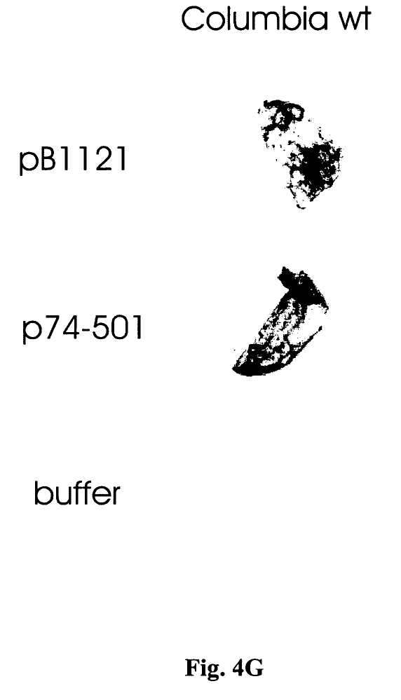

An operator sequence may consist of inverted repeat or palindromic sequences of a specified length. For example if the operator sequence is the Ros operator, it may comprise 9 or more nucleotide base pairs (see FIGS. 4 A and B) that exhibits the property of binding a DNA binding domain of a ROS repressor. A consensus sequence of a 10 base pair region including the 9 base pair DNA binding site sequence is WATDHWKMAR (SEQ ID NO: 23; FIG. 4B). The last nucleotide, “R”, of the consensus sequence is not required for ROS binding. Examples of operator sequences, which are not to be considered limiting in any manner, also include, as is the case with the ROS operator sequence from the virC or virD gene promoters, a ROS operator made up of two 11 bp inverted repeats separated by TTTA:

TATATTTCAATTTTATTGTAATATA (SEQ ID NO:17);

or the operator sequence of the ipt gene:

TATAATTAAAATATTAACTGTCGCATT (SEQ ID NO:18).

However, it is to be understood that analogs or variants of the operator sequence defined above may also be used, provided that they exhibit the property of binding a DNA binding domain. The Ros repressor has a DNA binding motif of the C₂H₂ zinc finger configuration. In the promoter of the divergent virC/virD genes of Agrobacterium tumefaciens, Ros binds to a 9 bp inverted repeat sequence in an orientation-independent manner (Chou et al., 1998). The Ros operator sequence in the ipt promoter also consists of a similar sequence to that in the virC/virD except that it does not form an inverted repeat (Chou et al., 1998). Only the first 9 bp are homologous to Ros box in virC/virD indicating that the second 9 bp sequence may not be a requisite for Ros binding. Accordingly, the use of Ros operator sequences or variants thereof that retain the ability to interact with Ros, as operator sequences to selectively control the expression of the first coding region, may be used as an operator sequence (40) as described herein.

It is to be understood that other repressor-operator combinations may be used, and that the Ros and Tet operator sequences are provided as non limiting examples only.

An operator sequence may be placed downstream, upstream, or upstream and downstream of the TATA box within a regulatory region. The operator sequences may also be placed within a promoter region as single binding elements or as tandem repeats. Furthermore, tandem repeats of an operator sequence can be placed downstream of the entire promoter or regulatory region and upstream of the first coding region. An operator sequence, or repeats of an operator sequence may also be positioned within untranslated or translated leader sequences, introns, or within the ORF (open reading frame) of the first coding region, if inserted in-frame.

The present invention provides a plant or portion thereof, capable of expressing both a first nucleotide sequence (10) and a second nucleotide sequence (50). The first nucleotide sequence comprising:

a first regulatory region (20) in operative association with a first coding region (30). The first coding region encodes a tag protein (35), and an operator sequence (40) capable of binding a repressor (95).

The second nucleotide sequence (50) comprising:

a second regulatory region (60) in operative association with a second coding sequence (70). The second coding region comprising a coding region of interest; and

a third regulatory region (80) in operative association with a third coding region (90). The third coding region encodes a repressor (95) capable of binding to the operator sequence (40) of the first nucleotide sequence (10). Binding of the repressor (95) to the operator sequence (40) reduces or inhibits expression of the first coding region (30).

The present invention also provides a plant or portion thereof, capable of expressing a first nucleotide sequence (10). The first nucleotide sequence comprising a first regulatory region (20) in operative association with a first coding region (30). The first coding region encodes a tag protein (35), and an operator sequence (40) capable of binding a repressor (95).

The present invention also provides a plant or a portion thereof, capable of expressing a second nucleotide sequence (50). The second nucleotide sequence comprising:

a second regulatory region (60) in operative association with a second coding sequence (70). The second coding region comprising a coding region of interest; and

a third regulatory region (80) in operative association with a third coding region (90). The third coding region encodes a repressor (95) capable of binding to the operator sequence (40) of the first nucleotide sequence (10). Binding of the repressor (95) to the operator sequence (40) reduces or inhibits expression of the first coding region (30).

By the term “repressor” (95, or 105, FIG. 3) it is meant a protein, peptide or fusion protein that, following binding to an operator sequence (40), down regulates expression of the first coding region (30), tag protein (35), or both, resulting in reduced mRNA, protein, or both synthesis. The repressor of the present invention may comprise any repressor known in the art, for example, but not limited to the ROS repressor, Tet repressor, Sin3, LacR and UMe6, or it may comprise a fusion protein, where the fusion protein comprises a repressor component, lacking a DNA binding domain, that is fused to a DNA binding domain of another protein. However, any repressor, a portion thereof, or fusion protein, which is capable of binding to an operator sequence, and down regulating expression of the first coding region (30), may be employed in the method of the present invention. Preferably, the repressor is the ROS repressor, or the Tet repressor, and the operator sequence comprises either a nucleotide sequence that binds the Ros repressor, or Tet repressor. Furthermore, it is preferred that the repressor comprises a nuclear localization signal.

By the term “fusion protein” it is meant a protein comprising two or more amino acid portions which are not normally found together within the same protein in nature and that are encoded by a single gene. Fusion proteins may be prepared by standard techniques in molecular biology known to those skilled in the art. It is preferred that at least one of the amino acid portions is capable of binding to the operator sequence (30) of the first nucleotide sequence (10).

By the term “binding” it is meant the reversible or non-reversible association of two components, for example the repressor and operator sequence. Preferably, the two components have a tendency to remain associated, but they may be capable of dissociation under appropriate conditions. These conditions may include, but are not limited to the addition of a third component which enhances dissociation of the bound components. For example, but not wishing to be limiting, the Tet repressor may be displaced from the Tet operator sequence by the addition of tetracycline.

The repressor (95), or a fusion protein comprising a repressor (105, FIG. 3) encoded by the third coding region (90, or 100, respectively) is capable of binding to the operator sequence (40) of the first nucleotide sequence (10). Binding of the repressor to the operator sequence reduces the level of mRNA, protein, or both mRNA and protein, encoded by the first coding region (30) for example a conditionally lethal coding region, compared to the level of mRNA, protein or both mRNA and protein produced in the absence of the repressor. Preferably, the repressor reduces the level of mRNA, protein or both mRNA and protein from about 25% to about 100%, more preferably about 50% to about 100%. Non-limiting examples of constructs encoding a repressor include p74-101 (FIGS. 9A, 11), p74-107 (FIG. 9E), p74-108 (FIG. 9F), p74-313 (FIG. 9A), p76-104, p75-103, p76-102 (also see Table 5, Examples)

The operator sequence (40) is located in proximity to the first coding region (30) encoding a tag protein (35), in a region which reduces transcription of the first coding region, when the operator sequence (40) is bound with a repressor (95). For example, but not wishing to be limiting, the operator sequence may be positioned between the first regulatory region (20) and the first coding region (30) so that when a repressor is bound to the operator sequence there is reduced transcription. Without wishing to be bound by theory, reduced transcription may arise from interference with transcription factor, polymerase, or both, binding, or to inhibit migration of the polymerase along the first coding region (30). The operator sequence may also be positioned in any location relative to the first coding region, provided that binding of the repressor to the operator sequence reduces expression of the first coding region. Preferably, binding of the repressor to the operator sequence reduces expression of the first coding region by about 25% to about 100%, more preferably by about 50% to about 100% of its original expression in the absence of the repressor protein. Detection of the expression product of the first coding region (30) may be determined using any suitable method, including but not limited to, Northern hybridization, S1 nuclease, array analysis, PCR, or other methods as would be known to one of skill in the art.

As an example, which is not to be considered limiting in any manner, the repressor and operator sequence employed in the method of the present invention may comprise the Ros repressor and Ros operator sequence. By “Ros repressor” it is meant any Ros repressor, analog or derivative thereof as known within the art that is capable of binding to an operator sequence. These include the Ros repressor as described herein, as well as other microbial Ros repressors, for example but not limited to RosAR (Agrobacterium radiobacter; Brightwell et al., 1995), MucR (Rhizobium meliloti; Keller M et al., 1995), and RosR (Rhizobium elti; Bittinger et al., 1997; also see Cooley et al. 1991; Chou et al., 1998; Archdeacon J et al. 2000; D'Souza-Ault M. R., 1993; all of which are incorporated herein by reference) and Ros repressors which have been altered at the DNA level for codon optimization, meaning the selection of appropriate DNA nucleotides for the synthesis of oligonucleotide building blocks, and their subsequent enzymatic assembly, of a structural gene or fragment thereof in order to approach codon usage within plants.

Alternatively, the repressor and operator sequence employed in the present invention may comprise the Tet repressor and Tet operator sequence. This system has been shown to function in stably transformed plants and transiently transformed plant protoplasts (Gatz et al., 1991; Gatz and Quail 1988, which are incorporated herein by reference).

The Tn 10-encoded Tet repressor comprises a 24 KDa polypeptide that binds as a dimer to a 19 base pair operator sequence (Hillen et al., 1984). The dimeric Tet repressor has a molecular mass of 47 kDa (Hillen et al., 1984). This molecular mass is less than the 45-60 kDa molecular mass required for passive diffusion into the nucleus via nuclear pores (Paine et al., 1975).

Examples of Tet repressors and operator sequences which may be employed in the present invention are described in the prior art, for example, but not wishing to be limiting, U.S. Pat. No. 5,917,122, which is herein incorporated by reference.

The present invention also contemplates a repressor which further comprises a nuclear localization signal such as, but not limited to SV40 localization signal, PKKKRKV (see Robbins et al., 1991; Rizzo, P. et al, 1999; which are incorporated herein by reference) in order to improve the efficiency of transport to the plant nucleus to facilitate the interaction with its respective operator sequence. Other possible nuclear localization signals that may be used include but are not limited to those listed in Table 1:

TABLE 1
nuclear localization signals

| | | | | |
|---|---|---|---|---|
| Nuclear Protein | Organism | NLS | SEQ ID NO: | Ref |

| | | | | |
|---|---|---|---|---|
| AGAMOUS | A | RienttnrqvtfcKRR | 36 | 1 |
| TGA-1 A | T | RRlaqnreaaRKsRIRKK | 37 | 2 |
| TGA-1B | T | KKRaRlvnresaqlsRqRKK | 38 | 2 |
| 02 NLS B | M | RKRKesnresaRRsRyRK | 39 | 3 |
| NIa | V | KKnqkhklkm-32aa-KRK | 40 | 4 |
| Nucleoplasmin | X | KRpaatkkagqaKKKKI | 41 | 5 |
| N038 | X | KRiapdsaskvpRKKtR | 42 | 5 |
| N 1/N2 | X | KRKteeesplKdKdaKK | 43 | 5 |
| Glucocorticoid receptor | M, R | RKclqagmnleaRKtKK | 44 | 5 |
| α receptor | H | RKclqagmnleaRKtKK | 45 | 5 |
| β receptor | H | RKclqagmnleaRKtKK | 46 | 5 |
| Progesterone receptor | C, H, Ra | RKccqagmvlggRKfKK | 47 | 5 |
| Androgen receptor | H | RKcyeagmtlgaRKIKK | 48 | 5 |
| p53 | C | RRcfevrvcacpgRdRK | 49 | 5 |

+A, Arabidopsis;
X, Xenopus;
M, mouse;
R, rat;
Ra, rabbit;
H, human;
C, chicken;
T, tobacco;
M, maize;
V, potyvirus.
References:
1, Yanovsky et al., 1990;
2, van der Krol and Chua, 1991;
3, Varagona et al., 1992;
4, Carrington et al., 1991;
5, Robbins et al., 1991.

Incorporation of a nuclear localization signal into the repressor of the present invention may facilitate migration of the repressor into the nucleus. Without wishing to be bound by theory, reduced levels of repressor (95) elsewhere within the cell may be important when the DNA binding portion of the repressor or fusion protein may bind analogue operator sequences within other organelles, for example within the mitochondrion or chloroplast. Furthermore, the use of a nuclear localization signal may permit the use of a less active promoter or regulatory region (80) to drive the expression of the third coding region (5), encoding the repressor (95) while ensuring that the concentration of the repressor remains at a desired level within the nucleus, and that the concentration of the repressor is reduced elsewhere in the cell.

The present invention also provides a method for the selection of a coding region of interest comprising, introducing the coding region of interest (the second coding region; 70) into a transformed plant that comprises the first nucleotide sequence (10), to produce a dual transgenic plant comprising both the first (10) and second (50) nucleotide sequences, and selecting for the dual transgenic plant by assaying for the presence of the tag protein (95). For example, which is not to be considered limiting, if the tag protein is a conditionally lethal protein, then expression of the tag protein may be determined by exposing the transformed plant and the dual transgenic plant to conditions that permit the conditionally lethal protein to become conditionally lethal, thereby reducing the growth, development, or killing, the transformed plant. For example, the plants may be provided with a substrate that is converted to a toxic product by the conditionally lethal protein, or the activity of the first regulatory region (20) may be induced resulting in the expression of a conditionally lethal protein that utilizes an endogenous substrate. Similarly, if the tag protein is a marker, for example but not limited to GFP, an enzyme, or an antibody, then the presence of the tag protein may be determined.

By “operatively linked” or “in operative association” it is meant that the particular sequences, for example a regulatory sequence and the coding region, interact either directly or indirectly to carry out their intended function, such as mediation or modulation of expression of the coding region. The interaction of operatively linked sequences may, for example, be mediated by proteins that in turn interact with the sequences.

By “regulatory region” or “regulatory element” it is meant a portion of nucleic acid typically, but not always, upstream of the protein coding region of a gene, which may be comprised of either DNA or RNA, or both DNA and RNA. When a regulatory region is active, and in operative association, or operatively linked, with a coding region of interest, this may result in expression of the coding region of interest. A regulatory element may be capable of mediating organ specificity, or controlling developmental or temporal gene or coding region activation. A “regulatory region” includes promoter elements, core promoter elements exhibiting a basal promoter activity, elements that are inducible in response to an external stimulus, elements that mediate promoter activity such as negative regulatory elements or transcriptional enhancers. “Regulatory region”, as used herein, also includes elements that are active following transcription, for example, regulatory elements that modulate gene expression such as translational and transcriptional enhancers, translational and transcriptional repressors, upstream activating sequences, and mRNA instability determinants. Several of these latter elements may be located proximal to the coding region.

In the context of this disclosure, the term “regulatory element” or “regulatory region” typically refers to a sequence of DNA, usually, but not always, upstream (5′) to the coding sequence of a structural gene, which controls the expression of the coding region by providing a binding site for RNA polymerase and/or other factors required for transcription to start at a particular site. However, it is to be understood that other nucleotide sequences, located within introns, or 3′ of the sequence may also contribute to the regulation of expression of a coding region of interest. An example of a regulatory element that provides for the recognition for RNA polymerase or other transcriptional factors to ensure initiation at a particular site is a promoter element. Most, but not all, eukaryotic promoter elements contain a TATA box, a conserved nucleic acid sequence comprised of adenosine and thymidine nucleotide base pairs usually situated approximately 25 base pairs upstream of a transcriptional start site. A promoter element comprises a basal promoter element, responsible for the initiation of transcription, as well as other regulatory elements (as listed above) that modify gene expression.

There are several types of regulatory regions, including those that are developmentally regulated, inducible or constitutive. A regulatory region that is developmentally regulated, or controls the differential expression of a gene under its control, is activated within certain organs or tissues of an organ at specific times during the development of that organ or tissue. However, some regulatory regions that are developmentally regulated may preferentially be active within certain organs or tissues at specific developmental stages, they may also be active in a developmentally regulated manner, or at a basal level in other organs or tissues within the plant as well.

An inducible regulatory region is one that is capable of directly or indirectly activating transcription of one or more DNA sequences or genes in response to an inducer. In the absence of an inducer the DNA sequences or genes will not be transcribed. Typically the protein factor that binds specifically to an inducible regulatory region to activate transcription may be present in an inactive form which is then directly or indirectly converted to the active form by the inducer. However, the protein factor may also be absent. The inducer can be a chemical agent such as a protein, metabolite, growth regulator, herbicide or phenolic compound or a physiological stress imposed directly by heat, cold, salt, or toxic elements or indirectly through the action of a pathogen or disease agent such as a virus. A plant cell containing an inducible regulatory region may be exposed to an inducer by externally applying the inducer to the cell or plant such as by spraying, watering, heating or similar methods. Inducible regulatory elements may be derived from either plant or non-plant genes (e.g. Gatz, C. and Lenk, I. R. P.,1998; which is incorporated by reference). Examples of potential inducible promoters include, but are not limited to, teracycline-inducible promoter (Gatz, C., 1997; which is incorporated by reference), steroid inducible promoter (Aoyama, T. and Chua, N. H., 1997; which is incorporated by reference) and ethanol-inducible promoter (Salter, M. G., et al, 1998; Caddick, M X, et al,1998; which are incorporated by reference) cytokinin inducible IB6 and CK11 genes (Brandstatter, I. and Kieber, J. 1,1998; Kakimoto, T., 1996; which are incorporated by reference) and the auxin inducible element, DR5 (Ulmasov, T., et al., 1997; which is incorporated by reference).

A constitutive regulatory region directs the expression of a gene throughout the various parts of a plant and continuously throughout plant development. Examples of known constitutive regulatory elements include promoters associated with the CaMV 35S transcript. (Odell et al., 1985), the rice actin1 (Zhang et al, 1991), actin2 (An et al., 1996), or tms2 (U.S. Pat. No. 5,428,147, which is incorporated herein by reference), and triosephosphate isomerase 1 (Xu et. al.,1994) genes, the maize ubiquitin 1 gene (Cornejo et al, 1993), the Arabidopsis ubiquitin 1 and 6 genes (Holtorf et al, 1995), the tobacco “t-CUP” promoter (WO/99/67389; U.S. Pat. No. 5,824,872), the HPL promoter (WO 02/50291), and the tobacco translational initiation factor 4A gene (Mandel et al, 1995). The term “constitutive” as used herein does not necessarily indicate that a gene under control of the constitutive regulatory region is expressed at the same level in all cell types, but that the gene is expressed in a wide range of cell types even though variation in abundance is often observed.

The regulatory regions of the first (10) and second (50) nucleotide sequences denoted above, may be the same or different. In an aspect of an embodiment of the method of the present invention, but not wishing to be limiting, the first regulatory region (20) of the first nucleotide sequence (10), and both the second regulatory region (60) and third regulatory region (80) of the second nucleotide sequence (50) are constitutively active. In an alternate aspect of an embodiment of the present invention, the first regulatory element (20 and third regulatory element (80) are constitutively active and the second regulatory element (60), which is operatively linked to, and controls the expression of, the coding region of interest (70) is inducible. The second regulatory element (60) may also be active during a specific developmental stage preceding, during, or following that of the activity of the first regulatory element (20). In this way the expression of the coding region of interest (70) may be repressed or activated as desired within a plant. The regulatory element (60) controlling expression of the second coding region (70) may be the same as the regulatory element (80) controlling expression of the coding region (90) encoding the repressor (95). Such a system ensures that both the second coding region (70) encoding the coding region of interest (70) and sequence encoding the repressor (90) are expressed in the same tissues, at similar times, or both.

By “coding region of interest” it is meant any nucleotide sequence that is to be expressed within a plant cell, tissue or entire plant. A coding region of interest may encode a protein of interest such as, but not limited to an industrial enzyme, protein supplement, nutraceutical, or a value-added product for feed, food, or both feed and food use. Examples of such proteins of interest include, but are not limited to proteases, oxidases, phytases, chitinases, invertases, lipases, cellulases, xylanases, enzymes involved in oil biosynthesis, etc.

Also, the coding region of interest may encode a pharmaceutically active protein, for example growth factors, growth regulators, antibodies, antigens, their derivatives useful for immunization or vaccination and the like. Such proteins include, but are not limited to, interleukins, insulin, G-CSF, GM-CSF, HPG-CSF, M-CSF or combinations thereof, interferons, for example, interferon-α, interferon-β, interferon-γ, blood clotting factors, for example, Factor VIII, Factor IX, or tPA or combinations thereof. If the coding region of interest encodes a product that is directly or indirectly toxic to the plant, then by using the method of the present invention, such toxicity may be reduced throughout the plant by selectively expressing the coding region of interest within a desired tissue or at a desired stage of plant development.

A coding region of interest may also encode one, or more than one protein that enhances plant growth or development, for example but not limited to, proteins involved with enhancing salt tolerance, drought resistance, or nutrient utilization, within a plant, or one, or more than protein that imparts herbicide or pesticide resistance to a plant.

A coding region of interest may also include a nucleotide sequence that encodes a protein involved in regulation of transcription, for example DNA-binding proteins that act as enhancers or basal transcription factors. Moreover, a nucleotide sequence of interest may be comprised of a partial sequence or a chimeric sequence of any of the above genes, in a sense or antisense orientation.

The coding region of interest or the nucleotide sequence of interest may be expressed in suitable plant hosts which are transformed by the nucleotide sequences, or genetic constructs, or vectors of the present invention. Examples of suitable hosts include, but are not limited to, agricultural crops including canola, Brassica spp., Arabidopsis, maize, tobacco, alfalfa, rice, soybean, pea, wheat, barley, sunflower, potato, tomato, and cotton, as well as horticultural crops and trees.

The first, second or third nucleotide sequences may further comprise a 3′ untranslated region. A 3′ untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3′ end of the mRNA precursor. Polyadenylation signals are commonly recognized by the presence of homology to the canonical form 5′-AATAAA-3′ although variations are not uncommon.

Examples of suitable 3′ regions are the 3′ transcribed, non-translated regions containing a polyadenylation signal of Agrobacterium tumor inducing (Ti) plasmid genes, such as the nopaline synthase (Nos gene) and plant genes such as the soybean storage protein genes and the small subunit of the ribulose-1,5-bisphosphate carboxylase (ssRUBISCO) gene.

The present invention also provides for vectors or chimeric constructs comprising the first nucleotide sequence (10), or the second nucleotide sequence. The chimeric gene construct of the present invention can also include further enhancers, either translation or transcription enhancers, as may be required. These enhancer regions are well known to persons skilled in the art, and can include the ATG initiation codon and adjacent sequences. The initiation codon must be in phase with the reading frame of the coding sequence to ensure translation of the entire sequence. The translation control signals and initiation codons can be from a variety of origins, both natural and synthetic. Translational initiation regions may be provided from the source of the transcriptional initiation region, or from the structural gene. The sequence can also be derived from the regulatory element selected to express the gene, and can be specifically modified so as to increase translation of the mRNA.

Also considered part of this invention are transgenic plants containing the chimeric construct comprising the first (10), second (50), or both the first and second nucleotide sequences, as described herein.

Methods of regenerating whole plants from plant cells are also known in the art. In general, transformed plant cells are cultured in an appropriate medium, which may contain selective agents such as antibiotics, where selectable markers are used to facilitate identification of transformed plant cells. Once callus forms, shoot formation can be encouraged by employing the appropriate plant hormones in accordance with known methods and the shoots transferred to rooting medium for regeneration of plants. The plants may then be used to establish repetitive generations, either from seeds or using vegetative propagation techniques.

The constructs of the present invention can be introduced into plant cells using Ti plasmids, Ri plasmids, plant virus vectors, direct DNA transformation, micro-injection, electroporation, etc. For reviews of such techniques see for example Weissbach and Weissbach (1988); Geierson and Corey, (1988); and Miki and Iyer (1997). For Arabidospsis see Clough and Bent (1998). The present invention further includes a suitable vector comprising the chimeric gene construct.

A non-limiting example of a first coding region (30) is the iaaH sequence. The first sequence (10) links the iaaH open reading frame (coding region), to a constitutive promoter (20) that has been altered to incorporate the DNA binding sites for a transcriptional repressor protein (the operator sequence (40)). When this construct is introduced into a plant, the resultant transgenic plant is sensitized to IAM exposure, or its analogues, as this chemical is converted to IAA causing aberrant cell growth and eventual death of the transgenic plant. This transgenic plant then serves as a platform line for subsequent transformations. The second construct (50) physically links the coding region of interest (70) to a third sequence (90) encoding a transcriptional repressor protein (95) whose respective DNA binding site (40) resides within the altered iaaH promoter (20) of the first construct (10). When introduced into the platform line the repressor protein (95) blocks expression of iaaH coding region (30) effectively desensitizing these cells to the actions of IAM, allowing such lines to grow in the presence of IAM.

As non-limiting examples of a first nucleotide sequence (10), several constitutive promoters (20) were modified to include DNA binding regions (40) recognizable by either the Tet or Ros repressor proteins (95) as indicated in Table 1 (see Examples). Each of the chimeric regulatory regions (comprising a regulatory region (20) and an operator sequence (40)) listed in Table 1 was fused, or operatively linked, to a coding region (30; reporter gene), in this case encoding the tag protein β-glucuronidase (GUS), and introduced into a plant, for example, Arabidopsis. When transgenic plant tissues were stained for GUS enzyme activity all of the regulatory regions were determined to be active and functioning in a normal constitutive manner. These plants are then used as platform plants.

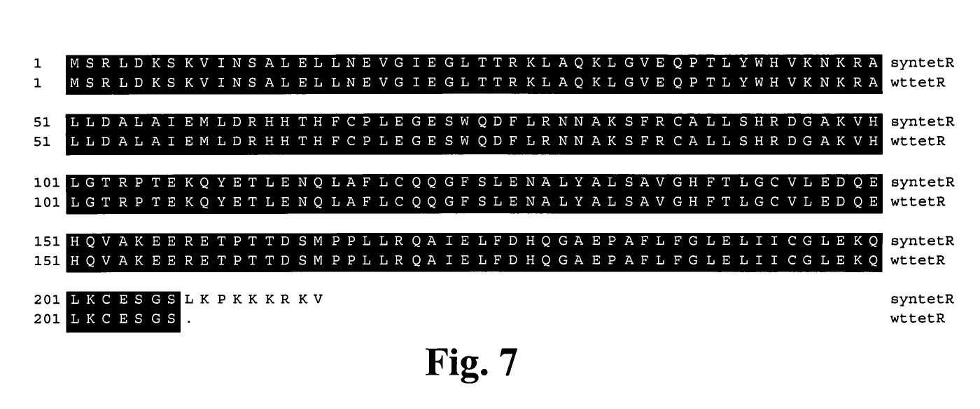

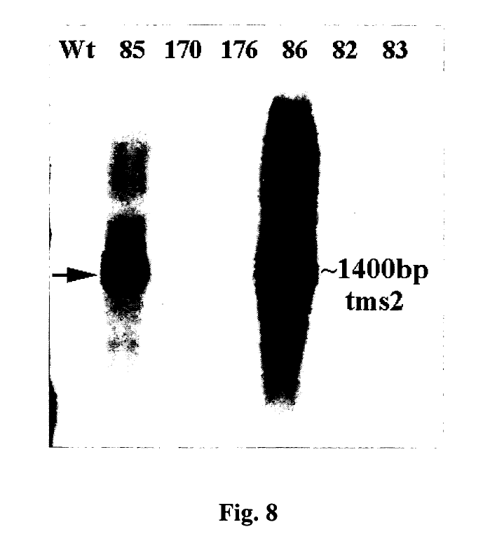

As an alternate example of a first nucleotide sequence, constructs comprising the iaaH gene (30) were prepared under the control of a constitutive promoter (20) modified to incorporate the DNA binding sites (40) for either the Tet or Ros repressor proteins (Table 3, see Examples). Northern blot analysis indicated that the modified actin2 promoters function in a normal constitutive manner to direct the expression of the iaaH gene (FIG. 8). The modified iaaH promoters also directed expression of the iaaH gene but at greatly reduced levels relative to the modified actin2 promoter. Plants treated with IAM exhibited abnormal growth and development, or death.

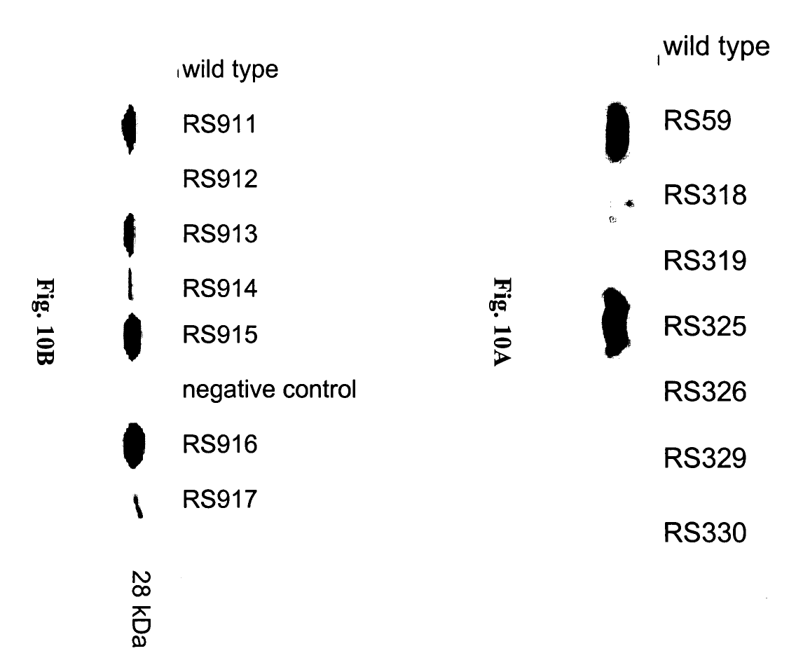

Wild type (wt) or optimized (syn) variants of either the Ros or tet repressor genes (90) were prepared (see Table 4, see Examples) and expressed in Arabidopsis plants under the control of constitutive promoters (80). Western blot analysis indicated that the Ros repressors were expressed effectively in the transgenic lines under the control of modified actin2, CaMV 35S and iaaH promoters (FIG. 10A). Expression of the synthetic Tet protein was also detected in plants transformed with a construct comprising a modified actin2 promoter to direct syn tet gene expression (FIG. 10B).

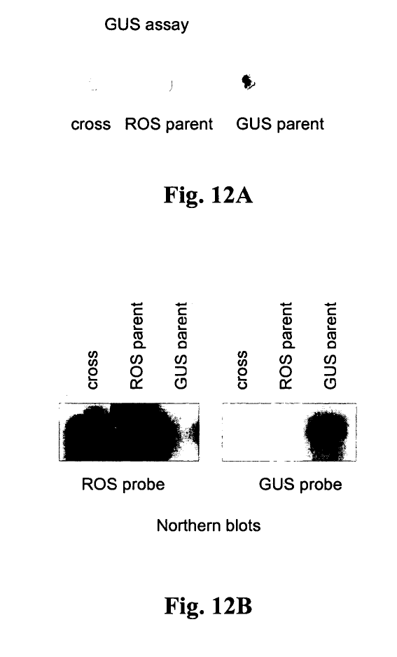

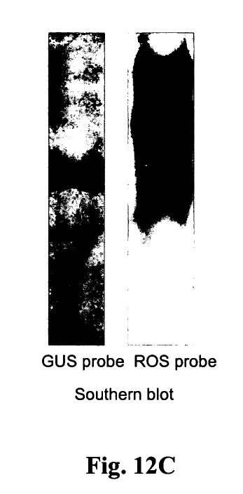

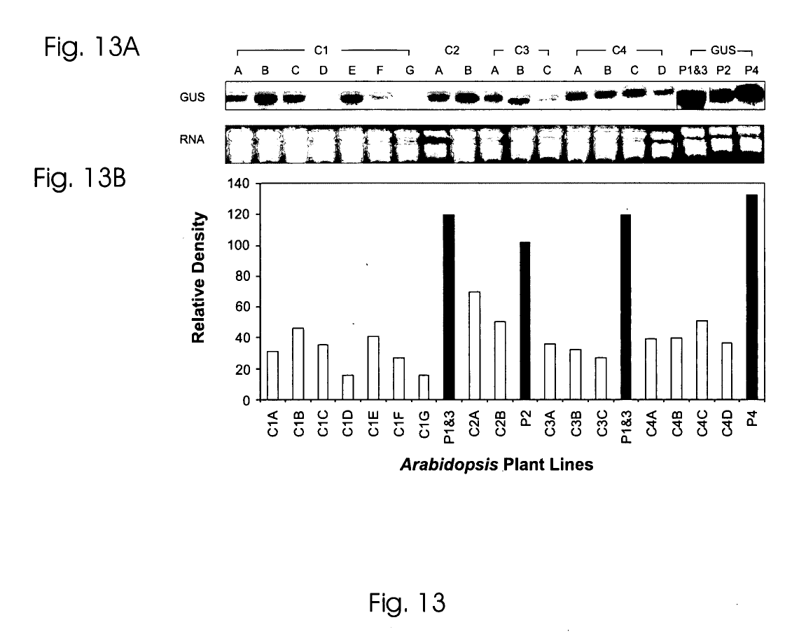

The ability of the repressor protein (95) to reduce expression of the tag protein (35), encoding in these examples either GUS or IAAH (30) and thus provide a marker for plant transformation was assessed. Plants expressing the first nucleotide sequence (10) were crossed with plants expressing the second nucleotide sequence (50), using standard techniques. As shown in FIGS. 12A, B and C, and in FIGS. 13A and B, the progeny of the crossed plants exhibited reduced or no tag protein expression.

Thus, in an aspect of an embodiment of the present invention, there is provided a method of selecting for a plant that comprises a coding region of interest (70). The method comprises,

i) providing a platform plant, or portion thereof, wherein the platform plant comprises a first nucleotide sequence (10) comprising, a first regulatory region (20) in operative association with a first coding region (30), and an operator sequence (40), the first coding region (30) encoding a tag protein (35);

ii) providing a second plant or portion thereof, the second plant comprising a second nucleotide (50) comprising, a second regulatory region (60) in operative association with a second coding region (70), and a third regulatory region (80) in operative association with a third coding region (90), the second coding region (70) comprising a coding region of interest, the third coding region (90) encoding a repressor (95);

iii) crossing the platform plant with the second plant to produce progeny

iv) selecting for dual transgenic plants expressing the second nucleotide sequence (50) within the progeny, by determining expression of the first coding region, the tag protein, or both, wherein the repressor protein (95) is capable of binding to the operator sequence (40) within the platform plant, thereby reducing or inhibiting expression of the first coding region.

The present invention also contemplates a method of selecting for transgenic plant cells comprising a coding region of interest (70), the method comprising,

i) providing a plant comprising a first nucleotide sequence (10), the first nucleotide sequence comprising,

a first regulatory region (20) in operative association with a first coding region (30), and an operator sequence (40), the first coding region (30) encoding a tag protein (35);

ii) transforming the platform plant with a second nucleotide sequence (50), the second nucleotide sequence comprising:

a second regulatory region (60) in operative association with a second coding region (70), and a third regulatory region (80) in operative association with a third coding region (90), to produce a dual transgenic plant, the second coding region comprises a coding region of interest, the third coding region encoding a repressor (95) capable of binding to the operator sequence (40) of the first nucleotide sequence (10) thereby inhibiting expression of the first coding region; and

iii) selecting for the dual transgenic plant by assaying for the expression of first coding region, the tag protein or both.

Furthermore, the method of the present invention also pertains to a method as just described above, wherein the first (10) and second (50) nucleotide sequences are introduced into a plant or plant cell plant in sequential steps so that the platform plant is prepared by transforming a plant with the first nucleotide sequence (10) followed by transforming the platform plant with the second nucleotide sequence (50), or the first (10) and second (50) nucleotide sequences are introduced into a plant or plant cell plant at the same time, within a single transforming step.

Alternate genetic constructs which may be employed in the method of the present invention are shown in FIG. 3. FIG. 3 shows a first nucleotide sequence (10) comprising a first regulatory region (20) in operative association with a first coding region (30) and an operator sequence (40) capable of binding a repressor (95) or fusion protein (105) and inhibiting production of the tag protein (35). Also shown in FIG. 3 is a second nucleotide sequence (50) comprising a second regulatory region (60) in operative association with a second nucleotide sequence (100) encoding a fusion protein (105). The second nucleotide sequence (100) comprises a nucleotide sequence (110) encoding a nucleotide sequence (120) encoding a coding region of interest fused to a nucleotide sequence encoding a repressor. Optionally, there may a linker sequence (130) inserted between the nucleotide sequence (120) encoding a coding region of interest and the nucleotide sequence (110) encoding a repressor. The fusion-protein (105), when bound via its repressor portion (108) to the operator sequence (40) of the first nucleotide sequence (10) inhibits production of the tag protein (35).

The fusion protein (105) may comprise a linker region (109) separating the repressor (108) from the protein of interest (107). Further, the linker region (109) may comprise an enzymatic cleavage sequence that is capable of being cleaved by an enzyme. For example, but not meant to be limiting in any manner, the linker region may comprise a thrombin cleavage amino acid sequence which may be cleaved by thrombin. The cleavage sequence may also be chemically cleaved using methods as known in the art. A cleavable linker permits the repressor portion of the fusion protein to be liberated from the protein of interest. However, other methods of separating the repressor and protein of interest are also contemplated by the present invention.

The fusion protein may also comprise an amino acid sequence to aid in purification of the fusion protein. Such amino acid sequences are commonly referred to in the art as “affinity tags”. An example of an affinity tag is a hexahistidine tag comprising six histidine amino acid residues. Any affinity tag known in the art may be used in the fusion protein of the present invention. Further, the fusion protein may comprise both linker sequences and affinity tags.

In embodiments of the present invention wherein the second nucleotide sequence (50) comprises a fusion protein, the fusion protein exhibits properties, for example but not limited to a size, to ensure that the fusion protein is capable of entering the nucleus, for example, diffusing through the nuclear pores, and binding the operator sequence. Preferably the fusion protein is less than about 100 kDa. Further, the fusion protein may additionally comprise a nuclear localization signal to enhance transport of the fusion protein into the nucleus and facilitate its interaction with the operator sequence.

The present invention also contemplates nucleotide sequences encoding proteins that have been optimized by changing codons to favor plant codon usage. In order to maximize expression levels of the first, second or third coding regions, the nucleic acid sequences of nucleotide sequences may be examined and the coding regions modified to optimize for expression of the gene in plants, for example using a codon optimization procedure similar to that outlined by Sardana et al. (1996), and synthetic sequences prepared. Assembly of synthetic first, second and third coding regions of this invention is performed using standard technology know in the art. The gene may be assembled enzymatically, within a DNA vector, for example using PCR, or prepared from ligation of chemically synthesized oligonucleotide duplex segments.

Assembly of the synthetic Ros repressor gene of this invention is performed using standard technology known in the art. The gene may be assembled enzymatically, within a DNA vector, for example using PCR, or synthesized from chemically synthesized oligonucleotide duplex segments. The synthetic gene is then introduced into a plant using methods known in the art. Expression of the gene may be determined using methods known within the art, for example Northern analysis, Western analysis, or ELISA.

A non-limiting example of a synthetic Ros repressor coding region comprising codons optimized for expression within plants is shown in FIG. 4C. However, it is to be understood that other base pair combinations may be used for the preparation of a synthetic Ros repressor gene, using the methods as known in the art to optimize repressor expression within a plant.

Schematic representations of constructs capable of expressing synthetic Ros or wild type Ros are shown in FIG. 4C. Southern analysis (FIG. 4D) of Arabidopsis plants that are transformed with constructs comprising the second nucleic acid sequence (50) of the present invention, expressing Ros repressor protein (95), indicates that both the wild type Ros and the synthetic Ros are integrated into the chromosome of Arabidopsis. Western blots shown in FIG. 4E demonstrate that both native Ros and synthetic Ros may be expressed within plants.

Similarly, stable integration and expression of the first nucleotide sequence of the present invention comprising a first coding region (30) in operative association with a regulatory region (20) which is in operative association with an operator sequence (40) is seen in FIG. 4D (Southern analysis) and FIG. 12A (GUS expression).

Crossing plants expressing the first nucleotide sequence (10) expressing the tag protein (35), and the second nucleotide sequence (50) expressing the repressor (95) resulted in reduced expression of the tag protein, in this case GUS activity (FIG. 12A), and GUS RNA (FIG. 12B). The results in FIG. 12A demonstrate that the tag protein, as indicated by GUS activity, is detected in the platform plant comprising the first nucleotide sequence (10; labeled as GUS parent in FIG. 12A). No tag protein is detected in the plant comprising the second nucleotide sequence (50), as this plant does not comprise or express the tag protein. Furthermore, no tag protein is evident in the progeny (labeled Cross in FIG. 12A) of the cross between the platform plant comprising the first nucleotide sequence (GUS parent) with that of the plant comprising the second nucleotide sequence (ROS parent). In this example, the parent plants each expressed either GUS or Ros RNA as expected (FIG. 12B), yet no GUS RNA was detected in the progeny arising from a cross between the ROS and GUS parents. Southern analysis of the progeny of the cross between the GUS and ROS parents indicates that the progeny plant from the cross between the ROS and GUS parent comprised genes encoding both GUS and Ros (FIG. 12C).

Similar results of the inhibition of tag protein expression from about 20 to about 95% inhibition (of the tag protein expression observed in the parental lines), is also observed in a variety of crosses made between platform plants expressing tag protein and plants expressing repressor as shown in FIGS. 13 A (GUS expression) and B (Ros expression; see Table 6 of the Examples, or the figure legend for a description of the crosses shown in FIG. 13). FIG. 13D shows quantification of the data of FIG. 13A (using a GUS probe) and further demonstrates that progeny of a cross between a plant expressing a first nucleotide sequence (10) and a plant expressing a second nucleotide sequence (50) exhibit reduced levels of expression of a first coding region (30).

These data demonstrate that expression of the tag protein (35) can be controlled using a repressor (95) as described herein, thereby providing a means to determine whether the second nucleic acid sequence (50) is expressed within a plant without requiring the use of a marker within the second nucleic acids sequence.

An aspect of the present invention therefore provides a plant selection strategy to identify and select plants cells, tissue or entire plants which comprise a coding region of interest (70). The plant selection strategy exemplified by the various aspects of embodiments discussed above need not be based on antibiotic resistance. Further, the plant selection strategy is benign to the transformed plant and confers no advantage to other organisms in the event of gene transfer. The present invention also provides genetic constructs which may be employed in plant selection strategies.

The above description is not intended to limit the claimed invention in any manner, furthermore, the discussed combination of features might not be absolutely necessary for the inventive solution.

A list of sequence identification numbers of the present invention is given in Table 2.

TABLE 2
List of sequence identification numbers.

| SEQ | | |
|---|---|---|
| ID | | Table/ |
| NO: | Description | Figure |
| 1 | Synthetic Ros optimized for plant expression (DNA) | |
| 2 | Synthetic Tet optimized for plant expression (DNA) | |
| 3 | Synthetic Ros (protein) | |
| 4 | Synthetic Tet (protein) | |
| 5 | Actin2 promoter sense primer | |
| 6 | Actin2 promoter anti-sense primer | |
| 7 | Ros sense primer | |
| 8 | Ros anti-sense primer | |
| 9 | iaaH sense primer | |
| 10 | iaaH anti-sense primer | |
| 11 | Tet-FI primer | |
| 12 | Tet-RI primer | |
| 13 | iaaH ORF sense primer | |
| 14 | iaaH ORF anti-sense primer | |
| 15 | Ros-OP1 | |
| 16 | Ros-OP2 | |
| 17 | Ros inverted repeat operator of virC/virD gene (DNA) | |
| 18 | Ros inverted repeat operator of ipt gene (DNA) | |
| 19 | Wild-type Ros (A. tumefaciens) (DNA) | |
| 20 | Wild-type Tet (A tumefaciens) (DNA) | |
| 21 | Wild-type Ros (protein) | |
| 22 | Wild-type Tet (protein) | |
| 23 | Consensus Ros operator sequence (DNA) | |
| 24 | SV40 NLS | |
| 25 | Ros-OPDS | |
| 26 | Ros-OPDA | |
| 27 | p74-315 sequence from EcoRV to ATG of GUS (DNA) | |
| 28 | Ros-OPUS | |
| 29 | Ros-OPUA | |
| 30 | p74-316 sequence from EcoRV to ATG of GUS (DNA) | |
| 31 | Ros-OPPS | |
| 32 | Ros-OPPA | |
| 33 | p74-309 sequence from EcoRV to ATG of GUS (DNA) | |
| 34 | p74-118 sequence from EcoRV to ATG of GUS (DNA) | |
| 35 | p74-117 sequence from EcoRV to ATG of GUS (DNA) | |
| 36 | AGAMOUS protein NLS | Table 1 |
| 37 | TGA-1A protein NLS | Table 1 |
| 38 | TGA-1B protein NLS | Table 1 |
| 39 | O2 NLS B protein NLS | Table 1 |
| 40 | NIa protein NLS | Table 1 |
| 41 | Nucleoplasmin protein NLS | Table 1 |
| 42 | NO38 protein NLS | Table 1 |
| 43 | N1/N2 protein NLS | Table 1 |
| 44 | Glucocorticoid receptor NLS | Table 1 |
| 45 | Glucocorticoid a receptor NLS | Table 1 |
| 46 | Glucocorticoid b receptor NLS | Table 1 |
| 47 | Progesterone receptor NLS | Table 1 |
| 48 | Androgen receptor NLS | Table 1 |
| 49 | p53 protein NLS | Table 1 |
| 50 | p74-114 sequence from EcoRV to ATG of GUS (DNA) | |
| 51 | synRos forward primer | |
| 52 | synRos reverse primer | |
| 53 | wtRos forward primer | |
| 54 | wtRos reverse primer | |
| 55 | Ros oligonucleotide for Southwestern | |
| 56 | Tet oligonucleotide for Southwestern | |
| 57 | VirC/VirD Ros operator (1) (DNA) | |
| 58 | VirC/VirD Ros operator (2) (DNA) | |
| 59 | Ipt Ros operator (1) (DNA) | |
| 60 | Ipt Ros operator (2) (DNA) | |
| 61 | Ros operator sequence (1) (DNA) | FIG. 4B |

The present invention will be further illustrated in the following examples. However it is to be understood that these examples are for illustrative purposes only, and should not be used to limit the scope of the present invention in any manner.

### EXAMPLES

### Example 1: Plant Material and Transformation Procedure

Plant Material

Wild type Arabidopsis thaliana, ecotype Columbia, seeds were germinated on RediEarth (W.R. Grace & Co.) soil in pots covered with window screens under green house conditions (˜25° C., 16 hr light). Emerging bolts were cut back to encourage further bolting. Plants were used for transformation once multiple secondary bolts had been generated.

Plant Transformation

Plant transformation was carried out according to the floral dip procedure described in Clough and Bent (1998). Essentially, Agrobacterium tumefaciens transformed with the construct of interest was grown overnight in a 100 ml Luria-Bertani Broth (10 g/L NaCl, 10 g/L tryptone, 5 g/L yeast extract) containing 50 mg/ml kanamycin. The cell suspension culture was centrifuged at 3000×g for 15 min. The pellet was resuspended in 1 L of the transformation buffer [sucRose (5%), Silwet L77 (0.05%)(Loveland Industries, Greeley, Co.)]. The above-ground parts of the Arabidopsis plants were dipped into the Agrobacterium suspension for ˜1 min and the plants were then transferred to the greenhouse. The entire transformation process was repeated twice more at two day intervals. Plants were grown to maturity and seeds collected. To select for transformants, seeds were surface sterilized by washing in 0.05% Tween 20 for 5 minutes, with 95% ethanol for 5 min, and then with a solution containing sodium hypochlorite (1.575%) and Tween 20 (0.05%) for 10 min followed by 5 washings in sterile water. Sterile seeds were plated onto either Pete Lite medium [20-20-20 Peter's Professional Pete Lite fertilizer (Scott) (0.762 g/l), agar (0.7%), kanamycin (50 μg/ml), pH 5.5] or MS medium [MS salts (0.5×)(Sigma), B5 vitamins (1×), agar (0.7%), kanamycin (50 μg/ml) pH 5.7]. Plates were incubated at 20° C., 16 hr light/8 hr dark in a growth room. After approximately two weeks, seedlings possessing green primary leaves were transferred to soil for further screening and analysis.

Northern Blot Hybridization

Northern blot analysis was carried out on total RNA extracted from plant leaves to determine the level of gene expression in the parental lines and crosses. Hybridization with [α-32P]dCTP-labeled probes was carried out for 16-20 h at 65° C. in 7% SDS, 1 mM EDTA, 0.5 M Na₂HPO₄ (pH 7.2). Membranes were washed once in a solution of 5% SDS, 1 mM EDTA, 40 mM Na₂HPO₄ (pH 7.2) for 30 min, followed by washing in 1% SDS, 1 mM EDTA, 40 mM Na₂HPO₄ (pH 7.2) for 30 min. The membranes were subjected to autoradiography using X-OMAT XAR5 film, and the intensity of bands measured using densitometer Quantity One Software (BioRad). The strength of the Northern blot bands was normalized by expressing it as a percentage of the density of the respective 28S rRNA band on the RNA gel.

Western Blotting

Total plant protein extracts are analyzed for the expression of the Ros protein using a polyclonal rabbit anti-Ros antibody. Chemiluminescent detection of antigen-antibody complexes is carried out with goat anti-rabbit IgG secondary antibody conjugated to horseradish peroxidase-conjugated (Bio-Rad Laboratories) in conjunction with ECL detection reagent (Amersham Pharamcia Biotech).

Antiserum Production

The ORF of wild type Ros (wtRos) was amplified by PCR using the two primers:

| | | |
|---|---|---|
| | BamHI | |
| forward primer: | 5′-GCG GAT CCG ATG ACG GAA ACT GCA TAC-3′ | (SEQ ID NO:7) |
| | HindIII | |
| reverse primer: | 5′-GCA AGC TTC AAC GGT TCG CCT TGC G-3′ | (SEQ ID NO:8) |

which have terminal BamHI and HindIII sites, respectively. The PCR fragment was cloned between the BamHI and HindIII sites of the Escherichia coli expression vector pTRCHisB (InVitrogen) as a fusion with the polyhistidine (HIS) tag to generate the plasmid pTRCHisB-Ros. This plasmid was used to transform E. coli XL1-Blue cells, and Ros expression was induced using 1 mM IPTG (isopropyl β-D-thiogalactopyranoside). Protein purification was carried out under denaturing conditions in 6 M urea using the His-Bind Kit, and the protein was renatured by dialysis in gradually reduced concentrations of urea according to the manufacturer's instructions (Novagen). Anti-Ros antiserum was generated in rabbits using standard methods (Harlow and Lane, 1988, which is incorporated herein by reference). Briefly, rabbits (New Zealand white) were injected with 50 mg of wtRos protein in Freud's complete adjuvant. Rabbits were boosted twice with 50 mg protein in Freud's incomplete adjuvant at two-week intervals and bled approximately five weeks after initial immunization. The serum was collected by clotting, followed by centrifugation and stored at −20° C.

The Tet gene is cloned from E. coli tn10 by PCR. The nucleotide sequence encoding the Tet protein is expressed in, and purified from, E. coli, and the Tet protein used to generate an anti-Tet antiserum in rabbits using standard methods (Harlow and Lane, 1988).

### Example 2: Genetic Constructs

A) Construction of the Second Nucleotide Sequence (50, FIG. 2) comprising Ros, Tet, Synthetic Ros and Synthetic Tet Repressor Genes

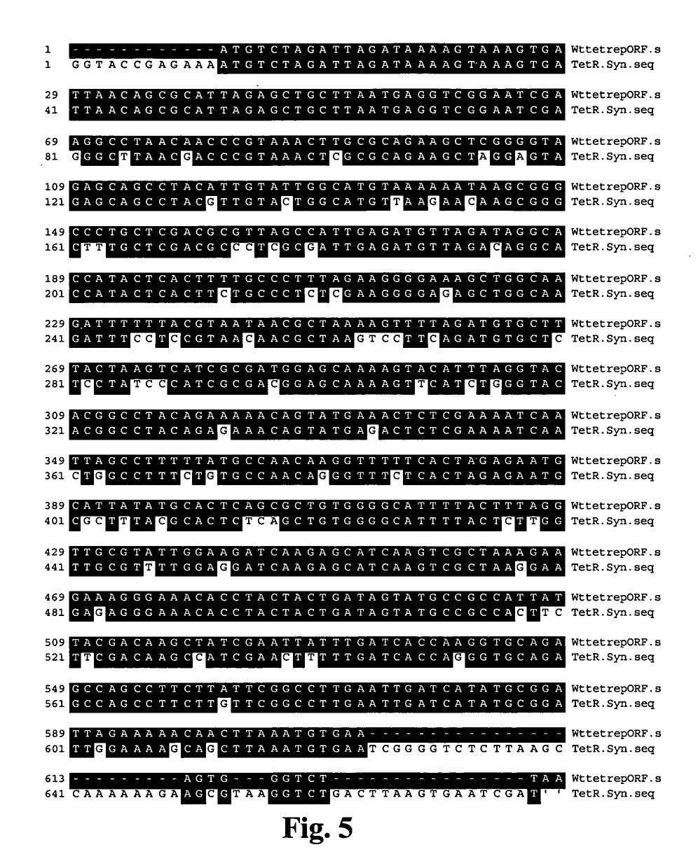

The Ros nucleotide sequence is derived from Agrobacterium tumefaciens (FIG. 4). The Tet nucleotide sequence (FIG. 5) is derived from the Escherichia coli tn10 transposon (Accession No. J01830).

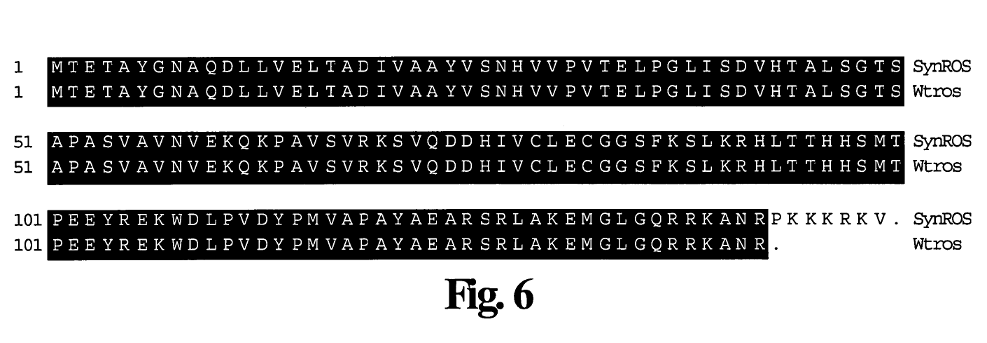

Analysis of the protein coding region of the Ros and Tet nucleotide sequences indicated that the codon usage may be altered to better conform to plant translational machinery. The protein coding region of the nucleotide sequence was therefore modified to optimize expression in plants (FIGS. 6 and 7). The nucleic acid sequences were examined and the coding regions modified to optimize for expression of the gene in plants, using a procedure similar to that outlined by Sardana et al. (1996). A table of codon usage from highly expressed genes of dicotyledonous plants was compiled using the data of Murray et al. (1989). The Ros and Tet nucleotide sequences were also modified to ensure localization of the repressors to the nucleus of plant cells, by adding the SV40 nuclear localization signal PKKKRKV (SEQ ID NO:24; Kalderon et al., 1984) at the 3′-end of the modified Ros gene upstream of the translation termination codon to enhance nuclear targeting. The modified synthetic gene was named synRos (FIG. 4C). 20

p74-101: Construct for The Expression of The Synthetic Ros Driven by The Actin2 Promoter (FIG. 9A, Table 5).

The actin2 promoter was PCR amplified from genomic DNA of Arabidopsis thaliana ecotype Columbia using the following primers:

| | | |
|---|---|---|
| | HindIII | |
| actin2 Sense primer | 5′-AAG CTT ATG TAT GCA AGA GTC AGC-3′ | (SEQ ID NO:5) |
| | SpeI | |
| actin2 anti-sense primer: | 5′-TTG ACT AGT ATC AGC CTC AGC CAT-3′ | (SEQ ID NO:6) |

The PCR fragment was cloned into pGEM-T-Easy. The 1.2 kbp HindIII/SpeI fragment of the actin2 promoter was then cloned into p74-313 as a HindIII/XbaI fragment replacing the CaMV 35S promoter.

p74-107: Construct for The Expression of The Wild Type Ros Driven by The CaMV 35S Promoter (FIG. 9E; Table 5)

The open reading frame of the wild type Ros gene was amplified by PCR using total genomic DNA of Agrobacterium tumefaciens 33970 and the following primers with built-in BamHI and HindIII sites were employed:

| | | |
|---|---|---|
| | BamHI | |
| Ros Sense primer: | 5′-GCG GAT CCG ATG ACG GAA ACT GCA TAC-3′ | (SEQ ID NO:7) |
| | HindIII | |
| Ros Anti-sense primer: | 5′-GCA AGC TTC AAC GGT TCG CCT TGC G-3′ | (SEQ ID NO:8) |

The PCR product was cloned into the BamHI/HindIII sites of the pGEX vector (Pharmacia), and was then excised from pGEX as a XhoI/BamHI fragment, and the XhoI site was blunt-ended using Klenow. The resulting fragment was cloned into the BamHI/EcoICR1 sites of pBI121 (Clontech).

p74-108: Construct for The Expression of The Synthetic Ros Repressor Driven by the iaaH Promoter (FIG. 9F; Table 5).

The iaaH promoter was PCR amplified from genomic DNA of Agrobacterium tumefaciens 33970 using the following two primers:

| | | |
|---|---|---|
| | HindIII | |
| iaaH Sense primer: | 5′-TGC GGA TGC ATA AGC TTG CTG ACA TTG CTA GAA AAG-3′ | (SEQ ID NO:9) |
| | BamHI | |
| iaaH Anti-sense primer: | 5′-CGG GGA TCC TTT CAG GGC CAT TTC AG-3′ | (SEQ ID NO:10) |

The 352 bp PCR fragment was cloned into the EcoRV site of pBluescript, and was then excised from pBluescript as a HindIII/BamHI fragment and sub-cloned into the HindIII/BamHI sites of p74-313 replacing the CaMV 35S promoter.

p74-313: Construct for The Expression of The Synthetic Ros Driven by The CaMV 35S Promoter (FIG. 9A; Table 5)

The open reading frame of the Ros repressor was re-synthesized to favor plant codon usage and to incorporate a nuclear localization signal, PKKKRKV (SEQ ID NO:24), at its carboxy-terminus as described above. The re-synthesized Ros was cloned into the BamHI-SacI sites of pUC19, and then was sub-cloned into pBI121 as a BamHI/SstI fragment replacing the GUS open reading frame in this vector.

p75-103: Construct for The Expression of The Synthetic Tet Driven by The actin2 Promoter (Table 5).

The actin2 promoter was PCR amplified from genomic DNA of Arabidopsis thaliana ecotype Columbia as described for p74-101 and cloned into pGEM-T-Easy. The 1.2 kbp HindIII/SpeI fragment of the actin2 promoter was then cloned into p76-102 as a HindIII/XbaI fragment replacing the CaMV 35S promoter.

p76-102: Construct for The Expression of The Synthetic Tet Driven by The CaMV 35S Promoter (Table 5).

The open reading of the Tet repressor was re-synthesized to favor plant codon usage and to incorporate a nuclear localization signal, PKKKRKV (SEQ ID NO:24), at its carboxy-terminus. The re-synthesized Tet was cloned into the KpnI/ClaI sites of pUC19, sub-cloned into pBluescript as a EcoRI/HindIII fragment, and then excised as a XbaI/HindIII where the HindIII cohesive end was blunt-ended by Klenow large fragment polymerase. The resulting fragment was then inserted into the XbaI/EcoICR1 sites of pBI121 replacing the GUS open reading frame in this vector.

p76-104: Construct for The Expression of The Synthetic Tet Gene Driven by the iaaH Promoter (Table 5).

The iaaH promoter was PCR amplified from genomic DNA of Agrobacterium tumefaciens 33970 using the following primers:

| | | |
|---|---|---|
| iaaH Sense primer: | 5′-TGC GGA TGC ATA AGC TTG CTG ACA TTG CTA GAA AAG-3′ | (SEQ ID NO:9) |
| iaaH Anti-sense primer: | 5′-CGG GGA TCC TTT CAG GGC CAT TTC AG-3′ | (SEQ ID NO:10) |

The 352 bp PCR fragment was cloned into the EcoRV site of pBluescript, sub-cloned into pGEM-7Zf(+), and then cloned into the HindIII/XbaI of p76-102 replacing the CaMV 35S promoter.

B) Construction of the First Nucleotide Sequence (10; FIG. 2) comprising Ros and Tet Operator Sequences (40) and a Coding Region (30) Encoding a Conditionally Lethal Tag Protein

p74-311: Construct for The Expression of The iaaH Gene Driven by the actin2 Promoter Containing a Tet Operator (Table 3).

The actin2 promoter was PCR amplified from genomic DNA of Arabidopsis thaliana ecotype Columbia as described for p74-101 and cloned into pGEM-T-Easy. Two complementary oligos, Tet-F1 and Tet-R1, with built-in BamHI and ClaI sites, and containing two Tet operators, were annealed together and then inserted into the actin2 promoter at the BglII/ClaI sites replacing the BglII/ClaI fragment. This modified promoter was inserted into pBI121 vector as a HindIII/BamHI fragment and designated p74-311.

| | | |
|---|---|---|
| | BamHI | |
| Tet-F1: | 5′-GAT CAC TCT ATC AGT GAT AGA GTG AAC TCT ATC AGT GAT AGA G-3′ | (SEQ ID NO:11) |
| | ClaI | |
| Tet-R1: | 5′-CGC TCT ATC ACT GAT AGA GTT CAC TCT ATC ACT GAT AGA GT-3′ | (SEQ ID NO:12) |

The iaaH open reading frame was PCR amplified from genomic DNA of Agrobacterium tumefaciens 33970 using the following two primers:

| | | |
|---|---|---|
| | XbaI | |
| iaaH ORF Sense primer: | 5′-GCT CTA GAA TGG TGC CCA TTA CCT CG-3′ | (SEQ ID NO:13) |
| | SstI | |
| iaaH ORF | 5′-GCG AGC TCA WAT GGC TTY TTC YAA TG-3′ | (SEQ ID NO:14) |
| Anti-sense primer: | | |

The 1387 bp PCR fragment was cloned into pGEM-T-Easy, sub-cloned into pBluescript, excised from pBluescript and inserted into the BamHI/SstI site of p74-311, thereby replacing the GUS ORF.

p74-503 Construct for The Expression of the iaaH Gene Driven by The actin2 Promoter Containing a Ros Operator (Table 4)

The actin2 promoter was PCR amplified from genomic DNA of Arabidopsis thaliana ecotype Columbia as described for p74-101 and cloned into pGEM-T-Easy. Two complementary oligos, Ros-OP1 (SEQ ID NO: 15) and Ros-OP2 (SEQ ID NO: 16), with built-in BamHI and ClaI sites, and containing two Ros operators, were annealed together and then inserted into the actin2 promoter at the BglII/ClaI sites replacing the BglII/ClaI fragment. This modified promoter was inserted into pBI121 vector as a HindIII/BamHI fragment. The GUS open reading frame was then excised and replaced with a BamHI/SstI iaaH open reading frame fragment obtained as described for p74-311.

| | | |
|---|---|---|
| | BamHI | |
| Ros-OP1: | 5′-GAT CCT ATA TTT CAA TTT TAT TGT AAT ATA GCT ATA TTT CAA | (SEQ ID NO: 15) |
| | TTT TAT TGT AAT ATA AT-3′ | |
| | ClaI | |
| | BamHI | |
| Ros-OP2: | 5′-CGA TTA TAT TAC AAT AAA ATT GAA ATA TAG CTA TAT TAC | (SEQ ID NO:16) |
| | AAT AAA ATT GAA ATA TAG-3′ | |
| | ClaI | |

p76-509: Construct for The Expression of The iaaH Gene Driven by the iaaH Promoter Containing a Ros Operator (Table 4).

The iaaH promoter was PCR amplified from genomic DNA of Agrobacterium tumefaciens 33970 as described for p76-104. Two complementary oligos, Ros-OP1 (SEQ ID NO: 15) and Ros-OP2 (SEQ ID NO: 16), containing two Ros operators, were annealed together and cloned into pGEM-7Zf(+) as a BamHI/ClaI fragment at the 3′ end of the iaaH promoter. This promoter/operator fragment was then sub-cloned into pBI121 as a HindIII/XbaI fragment, replacing the CaMV 35S promoter fragment. The GUS ORF was then excised and replaced with an XbaI/SstI iaaH open reading frame fragment. The tms2 ORF was PCR amplified from genomic DNA of Agrobacterium tumefaciens 33970 and cloned into pGEM-T-Easy as described for p74-311.

p76-510: Construct for The Expression of The iaaH Gene Driven by the iaaH Promoter Containing a Tet Operator (Table 4).

The tms2 promoter was PCR amplified from genomic DNA of Agrobacterium tumefaciens 33970 as described for p76-104. The 352 bp PCR fragment was cloned into the EcoRV site of pBluescript, and then sub-cloned into pGEM-7Zf(+).Two complementary oligos, Tet-F1 (SEQ ID NO: 11) and Tet-R1 (SEQ ID NO: 12), with built-in BamHI and ClaI sites, and containing two Tet operators, were annealed together and then inserted into the tms2 promoter at the BglII/ClaI sites. This modified promoter was inserted into pBI121 vector as a HindIII/XbaI fragment, thereby replacing the CaMV 35S promoter. The GUS open reading frame was then excised and replaced with an XbaI/SstI iaaH open reading frame fragment. The iaaH open reading frame was PCR amplified from genomic DNA of Agrobacterium tumefaciens 33970 and cloned into pGEM-T-Easy as described for p74-311.

C) Construction of the First Nucleotide Sequence (10; FIG. 2) comprising Ros and Tet Operator Sequences (40) and a Coding Region (30) Encoding a Tag Protein

p74-315: Construct for The Expression of GUS Gene Driven by a CaMV 35S Promoter Containing a Ros Operator Downstream of TATA Box (FIG. 9B; Table 3).

The BamHI-EcoRV fragment of CaMV 35S promoter in pBI121 is cut out and replaced with a similar synthesized DNA fragment in which the 25 bp immediately downstream of the TATA box were replaced with the Ros operator sequence:

TATATTTCAATTTTATTGTAATATA (SEQ ID NO: 17).

Two complementary oligos, Ros-OPDS (SEQ ID NO:25) and Ros-OPDA (SEQ ID NO:26), with built-in BamHI-EcoRV ends, and spanning the BamHI-EcoRV region of CaMV35S, in which the 25 bp immediately downstream of the TATA box are replaced with the ROS operator sequence (SEQ ID NO: 17), are annealed together and then ligated into the BamHI-EcoRV sites of CaMV35S.

| | | |
|---|---|---|
| Ros-OPDS: | 5′-ATC TCC ACT GAC GTA AGG GAT GAC GCA CAA TCC CAC TAT | (SEQ ID NO:25) |
| | CCT TCG CAA GAC CCT TCC TCT ATA TAA TAT ATT TCA ATT | |
| | TTA TTG TAA TAT AAC ACG GGG GAC TCT AGA G-3′ | |
| Ros-OPDA: | 5′-G ATC CTC TAG AGT CCC CCG TGT TAT ATT ACA ATA AAA | (SEQ ID NO:26) |
| | TTG AAA TAT ATT ATA TAG AGG AAG GGT CTT GCG AAG GAT | |
| | AGT GGG ATT GTG CGT CAT CCC TTA CGT CAG TGG AGA T-3′ | |

The p74-315 sequence from the EcoRV site (GAT ATC) to the first codon (ATG) of GUS is shown below (SEQ ID NO:27; TATA box—lower case in bold; the synthetic Ros sequence—bold caps; a transcription start site—ACA, bold italics; BamHI site—GGA TCC; and the first of GUS, ATG, in italics; are also indicated):

| | |
|---|---|
| 5′-GAT ATC TCC ACT GAC GTA AGG GAT GAC GCA CAA TCC CAC TAT CCT TCG | (SEQ ID NO:27) |
| CAA GAC CCT TCC TCt ata taA TAT ATT TCA ATT TTA TTG TAA TAT [symbol] | |
| GGG GAC TCT AGA GGA TCC CCG GGT GGT CAG TCC CTT ATG-3′ | |

p74-316: Construct for The Expression of GUS Driven by a CaMV 35S Promoter Containing a Ros Operator Upstream of TATA Box (FIG. 9A: Table 3).

The BamHI-EcoRV fragment of CaMV 35S promoter in pBI121 is cut out and replaced with a similar synthesized DNA fragment in which the 25 bp immediately upstream of the TATA box are replaced with the ROS operator sequence (SEQ ID NO: 17). Two complementary oligos, Ros-OPUS (SEQ ID NO:28) and Ros-OPUA (SEQ ID NO:29), with built-in BamHI-EcoRV ends, and spanning the BamHI-EcoRV region of CaMV35S, in which the 25 bp immediately upstream of the TATA box were replaced with a Ros operator sequence (SEQ ID NO: 17), are annealed together and then ligated into the BamHI-EcoRV sites of CaMV35S.

| | | |
|---|---|---|
| Ros-OPUS: | 5′-ATC TCC ACT GAC GTA AGG GAT GAC GCA CAA TCT ATA TTT | (SEQ ID NO:28) |
| | CAA TTT TAT TGT AAT ATA CTA TAT AAG GAA GTT CAT TTC | |
| | ATT TGG AGA GAA CAC GGG GGA CTC TAG AG-3′ | |
| Ros-OPUA: | 5′-G ATC CTC TAG AGT CCC CCG TGT TCT CTC CAA ATG AAA | (SEQ ID NO:29) |
| | TGA ACT TCC TTA TAT AGT ATA TTA CAA TAA AAT TGA AAT | |
| | ATA GAT TGT GCG TCA TCC CTT ACG TCA GTG GAG AT-3′ | |

The p74-316 sequence from the EcoRV site (GAT ATC) to the first codon (ATG) of GUS is shown below (SEQ ID NO: 30; TATA box—lower case in bold; the synthetic Ros sequence—bold caps; a transcription start site—ACA, bold italics; BamHI site—GGA TCC; the first codon of GUS, ATG—italics, are also indicated):

| | |
|---|---|
| 5′-GAT ATC TCC ACT GAC GTA AGG GAT GAC GCA CAA TCT ATA TTT CAA | (SEQ ID NO:30) |
| TTT TAT TGT AAT ATA Cta tat aAG GAA GTT CAT TTC ATT TGG AGA [symbol] | |
| GGG GGA CTC TAG AGG ATC CCC GGG TGG TCA GTC CCT TAT G-3′ | |

p74-117 Construct for The Expression of GUS Driven by a CaMV 35 S Promoter Containing One Ros Operator Upstream of the TATA Box and Two Ros Operators Downstream of TATA Box

The BamHI-EcoRV fragment of CaMV 35S promoter in pBI121 was cut out and replaced with a similar synthesized DNA fragment in which a region up and downstream of the TATA box was replaced with three Ros operator sequences (SEQ ID NO: 17). The first of the three synthetic Ros operator sequences is positioned 25 bp immediately upstream of the TATA box (see SED ID NO:35). The other two Ros operator sequences are located downstream of the transcriptional start site (ACA). These downstream Ros operator sequences were prepared using two complementary oligos with built-in BamHI-EcoRV ends, as described above (Ros-OPUS, SEQ ID NO:28, and Ros-OPUA, SEQ ID NO:29) which were annealed together and ligated into the BamHI-EcoRV sites of CaMV 35S.

The p74-117 sequence from the EcoRV site (GAT ATC) to the first codon (ATG) of GUS is shown below (SEQ ID NO: 35; TATA box—lower case in bold: the synthetic ROS sequence—bold caps; a transcription start site—ACA, bold italics: BamHI site—GGA TCC; the first codon of GUS, ATG—italics, are also indicated);

| | |
|---|---|
| 5′-GAT ATC TCC ACT GAC GTA AGG GAT GAC GCA CAA TCT ATA TTT CAA | (SEQ ID NO:35) |
| TTT TAT TGT AAT ATA Cta tat aAG GAA GTT CAT TTC ATT TGG AGA [symbol] | |
| GGG GGA CTC TAG AGG ATC CTA TAT TTC AAT TTT ATT GTA ATA TAG GTA | |
| TAT TTC AAT TTT ATT GTA ATA TAA TCG ATT TCG AAC CCG GGG TAC CGA | |
| ATT CCT CGA GTC TAG AGG ATC CCC GGG TGG TCA GTC CCT TAT G-3′ | |

p74-309: Construct for The Expression of GUS Driven by a CaMV 35S Promoter Containing Ros Operators Upstream and Downstream of TATA Box (FIG. 9C; Table 3).

The BamHI-EcoRV fragment of CaMV 35S promoter in pBI121 is cut out and replaced with a similar synthesized DNA fragment in which the 25 bp immediately upstream and downstream of the TATA box were replaced with two Ros operator sequences (SEQ ID NO:17). Two complementary oligos, Ros-OPPS (SEQ ID NO:31) and Ros-OPPA (SEQ ID NO:32), with built-in BamHI-EcoRV ends, and spanning the BamHI-EcoRV region of CaMV 35S, in which the 25 bp immediately upstream and downstream of the TATA box are replaced with two ROS operator sequences, each comprising the sequence of SEQ ID NO:25 (in italics, below), are annealed together and ligated into the BamHI-EcoRV sites of CaMV35S.

| | | |
|---|---|---|
| Ros-OPPS: | 5′-ATC TCC ACT GAC GTA AGG GAT GAC GCA CAA TCT ATA TTT CAA | (SEQ ID NO:31) |
| | TTT TAT TGT AAT ATA CTA TAT AAT ATA TTT CAA TTT TAT TGT AAT | |
| | ATA ACA CGG GGG ACT CTA GAG-3′ | |
| Ros-OPPA: | 5′-G ATC CTC TAG AGT CCC CCG TGT TAT ATT ACA ATA AAA TTG AAA | (SEQ ID NO:32) |
| | TAT ATT ATA TAG TAT ATT ACA ATA AAA TTG AAA TAT AGA TTG TGC GTC | |
| | ATC CCT TAC GTC AGT GGA GAT-3′ | |

The p74-309 sequence from the EcoRV site (GAT ATC) to the first codon (ATG) of GUS is shown below (SEQ ID NO:33; TATA box—lower case in bold; two synthetic Ros sequence—bold caps; a transcription start site—ACA, bold italics; BamHI site—GGA TCC; the first codon of GUS, ATG—italics, are also indicated):

| | |
|---|---|
| 5′-GAT ATC TCC ACT GAC GTA AGG GAT GAC GCA CAA TCT ATA TTT CAA | (SEQ ID NO:33) |
| TTT TAT TGT AAT ATA Cta tat aAT ATA TTT CAA TTT TAT TGT AAT ATA | |
| [symbol] CGG GGG ACT CTA GAG GAT CCC CGG GTG GTC AGT CCC TTA TG-3′ | |

p76-508: Construct for The Expression of The GUS Gene Driven by the tms2 (iaaH) Promoter Containing a Ros Operator (FIG. 9D; Table 3).

The tms2 (iaah) promoter is PCR amplified from genomic DNA of Agrobacterium tumefaciens 33970 using the following primers:

| | | |
|---|---|---|
| iaaH sense primer: | 5′-TGC GGA TGC ATA AGC TTG CTG ACA TTG CTA GAA AAG-3′ | (SEQ ID NO:9) |
| iaaH anti-sense primer: | 5′-CGG GGA TCC TTT CAG GGC CAT TTC AG-3′ | (SEQ ID NO:10) |

The 352 bp PCR fragment is cloned into the EcoRV site of pBluescript, and sub-cloned into pGEM-7Zf(+). Two complementary oligos, Ros-OP1 (SEQ ID NO: 15) and Ros-OP2 (SEQ ID NO:16), containing two Ros operators (in italics, below), are annealed together and cloned into pGEM-7Zf(+) as a BamHI/ClaI fragment at the 3′ end of the tms2 promoter. This promoter/operator fragment is then sub-cloned into pBI121 as a HindIII/XbaI fragment, replacing the CaMV 35S promoter fragment.

| | | |
|---|---|---|
| Ros-OP1: | 5′-GAT CCT ATA TTT CAA TTT TAT TGT AAT ATA GCT ATA TTT CAA TTT | (SEQ ID NO:15) |
| | TAT TGT AAT ATA AT-3′ | |
| Ros-OP2: | 5′-CGA TTA TAT TAG AAT AAA ATT GAA ATA TAG CTA TAT TAC AAT | (SEQ ID NO:16) |
| | AAA ATT GAA ATA TAG-3′. | |

As a control, p76-507 comprising a tms2 promoter (without any operator sequence) fused to GUS, is also prepared.

p74-501: Construct for The Expression of The GUS Gene Driven by The actin2 Promoter Containing a Ros Operator (FIG. 9A: Table 3).

The actin2 promoter is PCR amplified from genomic DNA of Arabidopsis thaliana ecotype Columbia using the following primers:

| | | |
|---|---|---|
| actin2 Sense primer: | 5′-AAG CTT ATG TAT GCA AGA GTC AGC-3′ | (SEQ ID NO:5) |
| actin2 Anti-sense primer: | 5′-TTG ACT AGT ATC AGC CTC AGC CAT-3′ | (SEQ ID NO:6) |

The PCR fragment is cloned into pGEM-T-Easy. Two complementary oligos, Ros-OP 1 (SEQ ID NO: 15) and Ros-OP2 (SEQ ID NO: 16), with built-in BamHI and ClaI sites, and containing two Ros operators, are annealed together and inserted into the actin2 promoter at the BglII/ClaI sites replacing the BglII/ClaI fragment. This modified promoter is inserted into pBI121 vector as a HindIII/BamHI fragment.

p74-118 Construct for The Expression of GUS Driven by a CaMV 35S Promoter Containing Three RosOperators Downstream of TATA Box (FIG. 9A; Table 3).

The BamH1-EcoRV fragment of CaMV 35S promoter in pBI121 is cut out and replaced with a similar synthesized DNA fragment in which a region downstream of the TATA box was replaced with three Ros operator sequences (SEQ ID NO:35). The first of the three synthetic Ros operator sequences is positioned immediately of the TATA box, the other two Ros operator sequence are located downstream of the trasncriptional start site (ACA). Two complementary oligos with built-in BamHI-EcoRV ends were prepared as describe above for the other constructs were annealed together and ligated into the BamHI-EcoRV sites of CaMV35S.

The p74-118 sequence from the EcoRV site (GAT ATC) to the first codon (ATG) of GUS is shown below (SEQ ID NO:34; TATA box—lower case in bold; three synthetic Ros sequence—bold caps; a transcription start site—ACA, bold italics; BamHI site—GGA TCC; the first codon of GUS, ATG—italics, are also indicated):

| | |
|---|---|
| 5′-GAT1 ATC TCC ACT GAC GTA AGG GAT GAC GCA CAA TCC CAC TAT CCT TCG | (SEQ ID NO:34) |
| CAA GAC CCT TCC TCt ata taA TAT ATT TCA ATT TTA TTG TAA TAT [symbol] | |
| GGG GAC TCT AGA GGA TCC TAT ATT TCA ATT TTA TTG TAA TAT AGC TAT | |
| ATT TCA ATT TTA TTG TAA TAT AAT CGA TTT CGA ACC CGG GGT ACC GAA | |
| TTC CTC GAG TCT AGA GGA TCC CCG GGT GGT CAG TCC CTT ATG-3′ | |

As a control, p75-101, comprising an actin2 promoter (without any operator sequence) fused to GUS, is also prepared.

The various constructs are introduced into Arabidopsis, as described above, and transgenic plants are generated. Transformed plants are verified using PCR or Southern analysis. FIG. 4D show Southern analysis of transgenic plants comprising a first nucleic acid, for example, p74-309 (35S-2X Ros operator sequence-GUS, FIG. 9C).

p74-114: Construct for The Expression of GUS Driven by a CaMV 35S Promoter Containing One Ros Operator Upstream and Three Ros Operators Downstream of TATA Box.

In order to construct p74-114 (see FIG. 12B) the BamHI-EcoRV fragment of CaMV 35S promoter in pBI121 is cut out and replaced with a similar synthesized DNA fragment in which a region upstream and downstream of the TATA box was replaced with four Ros operator sequences (SEQ ID NO: 17). The first of the four synthetic Ros operator sequences is positioned 25 bp immediately upstream of the TATA box. The second of the four synthetic Ros operator sequences is positioned 25 bp immediately downstream of the TATA box. The other two Ros operator sequences are located downstream of the transcriptional start site (ACA). Two complementary oligos (SEQ ID NO:31 and 32) with built-in BamHI-EcoRV ends were prepared as described above for the other constructs, were annealed together and ligated into the BamHI-EcoRV sites of CaMV 35S. The p74-114 sequence from the EcoRV site (GAT ATC) to the first codon (ATG) of GUS is shown below (SEQ ID NO:50); TATA box—lower case in bold: the synthetic Ros sequence—bold caps; a transcription start site—ACA, bold italics: BamHI site—GGA TCC; the first codon of GUS, ATG—italics, are also indicated);

| | |
|---|---|
| 5′-GAT ATC TCC ACT GAC GTA AGG GAT GAC GCA CAA TCT ATA TTT CAA | (SEQ ID NO:50) |
| TTT TAT TGT AAT ATA Cta tat aAT ATA TTT CAA TTT TAT TGT AAT ATA | |
| [symbol] ACA CGG GGG ACT CTA GAG GAT CCT ATA TTT CAA TTT TAT TGT AAT ATA | |
| GCT ATA TTT CAA TTT TAT TGT AAT ATA ATC GAT TTC GAA CCC GGG GTA | |
| CCG AAT TCC TCG AGT CTA GAG GAT CCC CGG GTG GTC AGT CCC TTA TG-3′ | |

### Example 3

GUS Expression Assays on Reporter Transgenic Lines

In order to assess the activity of the modified regulatory regions, the level of expression of the GUS gene is assayed. Leaf tissues (approximately 10 mg) from putative positive transformants are placed into a microtitre plate containing 100 μl of GUS staining buffer (100 mM KPO₄, 1 mM EDTA, 0.5 mM K-ferricyanide, 0.5 mM K-ferrocyanide, 0.1% Triton X-100, 1 mM 5-bromo-4-chloro-3-indolyl glucuronide), and vacuum-infiltrated for one hour. The plate is covered and incubated at 37° C. overnight. Tissues are destained when necessary using 95% ethanol and color reaction is evaluated either visually or with a microscope.

For the modified 35S promoter, 45 lines had high GUS expression levels. These include 15 lines containing the Ros operator upstream of the TATA box, 24 lines containing the Ros operator downstream of the TATA box and six lines containing the Ros operator upstream and downstream of the TATA box. Using the actin2 promoter, 8 lines containing the Ros operator displayed high levels of GUS activity. An example of GUS expression in a plant transformed with p74-501 (actin2-2xRos operator sequence-GUS) is shown in FIG. 4G.

Single copy transformants expressing various levels of GUS activity are used for crossing with repressor lines, expressing the second nucleic acid sequence prepared in Example 2, as outlined in Example 5.

SynRos Protein Expression in Arabidopsis

Transgenic A. thaliana lines possessing constructs for the expression of wtRos and synRos under the control of the CaMV35S promoter were generated to determine whether codon optimization resulted in improved expression of synRos as compared to wtRos. Western blot analysis of these lines using ROS polyclonal antibodies (data not shown) revealed an overall improvement in the expression level of synRos compared to that of the wtRos. Of the 35 plants having the wtRos contruct, expression was detected in only nine plants, three of which expressed moderate levels of ROS and six only very low levels. In contrast, 18 of 53 plants containing the synRos construct exhibited comparatively higher levels of Ros expression ranging from moderate to strong.

Levels of Ros protein, both wild type Ros (wtRos), for example p74-107 (35S-wtRos; FIG. 9E), and synthetic Ros, for example p74-101 (actin2-synRos; FIG. 9A), produced in the transgenic plants is determined by Western blot analysis using a Ros polyclonal antibody (FIG. 4F).

Transient Expression of the wtRos and synRos Fusion Proteins

The open reading frames (ORF) of synRos and wtRos (FIG. 4c) were amplified by PCR using the following primers having terminal BamHI and SacI sites (underlined):

| | | |
|---|---|---|
| synRos forward: | 5′-GCG GAT CCA TGA CTG AGA CTG CTT ACG GTA ACG-3′ | (SEQ ID NO:51) |
| synRos reverse: | 5′-GCG AGC TCG ACC TTA CGC TTC TTT TTT GG-3′ | (SEQ ID NO:52) |
| wtRos forward: | 5′-CG GGA TCC ATG ACG GAA ACT GCA TAC-3′ | (SEQ ID NO:53) |
| wtRos reverse: | 5′-GCG AGC TCA CGG TTC GCC TTG CGG-3′ | (SEQ ID NO:54) |

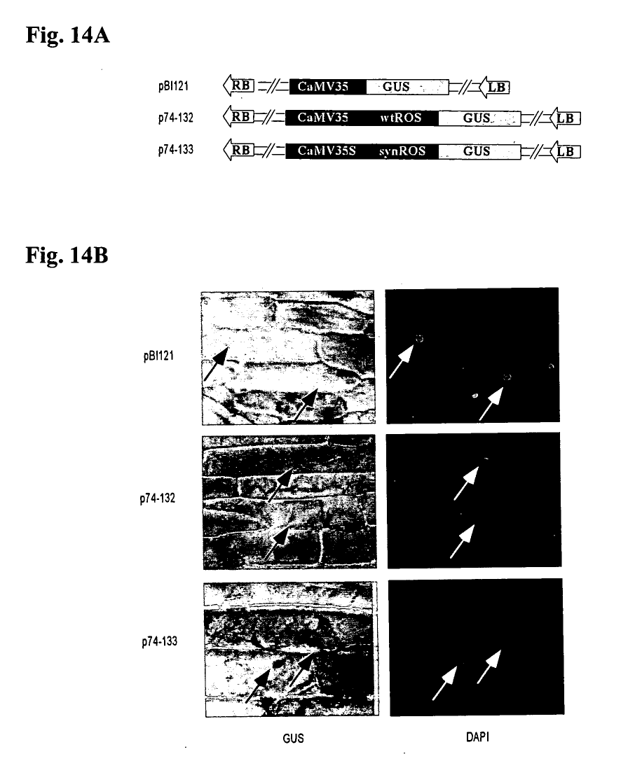

The amplified fragments were cloned between the BamHI-SacI sites of a derivative of vector CB301 (Gao et al., 2003) to generate constructs p74-133 and p74-132, which contain synRos-GUS and wtRos-GUS in-frame fusions, respectively, under the control of the CaMV35S promoter (FIG. 14). Onion epidermal layers were vacuum infiltrated with a culture of A. tumefaciens GV3101 pMP90 prepared as described by Kapila et al. (1997) with a few modifications. Briefly, the inner epidermal layers were peeled, placed into a bacterial culture containing p74-133, p74-132, or pBI121 for GUS expression only (BD Biosciences Clontech), and subjected to a vacuum of 85 kPa for 20 min. After incubation at 22° C. under 16 h light for three to five days, the tissues were placed into GUS staining solution [100 mM potassium phosphate buffer (pH 7.4), 1 mM EDTA, 0.5 mM K₃Fe(CN)₆, 0.5 mM K₄Fe(CN)₆, 0.1% Triton X-100, 1 mM 5-bromo-4-chloro-3-indolyl-β-D-glucuronide], vacuum infiltrated for 20 min at 85 kPa and incubated overnight at 37° C. To determine the location of nuclei, tissues were stained with 5 μg/ml DAPI (4′, 6-diamidino-2-phenylindole) (Varagona et al., 1991) and viewed under a Zeiss Photoscope III microscope using both fluorescence and differential interference contrast microscopy.

GUS localization in onion epidermal cell layers was analysed. GUS activity was observed exclusively in the cytoplasm of cells transformed with either the wtRos-GUS fusion or GUS alone (FIG. 14B). In contrast, GUS activity was localized in the nuclei of cells transformed with the synRos-GUS fusion construct, indicating that the inclusion of an SV40 nuclear targeting signal directs nuclear localization of the Ros protein.

Protein-DNA Interaction Analysis

The interaction of Ros with DNA sequences was examined using a modified Southwestern procedure. Briefly, double or single stranded DNA oligonucleotides were spotted onto Hybond-N membranes (Amersham Biosciences). The following oligonucleotides were used:

| | |
|---|---|
| Ros operator (underlined) | |
| 5′-ATC TCC ACT GAC GTA AGG GAT GAC GCA CAA TCT ATA TTT CAA TTT TAT | (SEQ ID NO:55) |
| TGT AAT ATA CTA TAT AAT ATA TTT CAA TTT TAT TGT AAT ATA ACA CGG | |
| GGG ACT CTA GAG-3′ | |
| tetR operator (underlined) | |
| 5′-GAT CAC TCT ATC AGT GAT AGA GTG AAC TCT ATC AGT GAT AGA G-3′ | (SEQ ID NO:56) |

The membranes were blocked in 10% skim milk in TBST [20 mM Tris (pH 7.5), 150 mM NaCl, 0.05% Tween 20] and the blot incubated with ˜100 μg of re-natured wtRos protein in 10% milk in TBST at room temperature for 2 hr. The membrane was washed three times in TBST and the protein-DNA complex detected using a polyclonal rabbit anti-wtRos antiserum. Chemiluminescent detection of antigen-antibody complexes was carried out with goat anti-rabbit IgG secondary antibody conjugated to horseradish peroxidase (Bio-Rad Laboratories) in conjunction with ECL detection reagent (Amersham Biosciences).

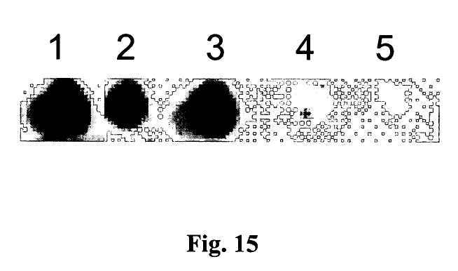

As shown in FIG. 15, wtRos expressed in E. coli bound to double stranded as well as single stranded Ros operators in both orientations, but not to control DNA representing two single stranded tandem tetR operators in the sense and anti-sense orientations.

### Example 4

Expression of GUS Gene in Arabidopsis

Several constitutive promoters were modified to include DNA binding regions recognizable by either the Tet or Ros repressor proteins (Table 3).

TABLE 3
Reporter Constructs (the first nucleotide sequence, 10, FIG. 2)

| | Base | | |
|---|---|---|---|
| Name | Promoter* | Operator** | Reporter |
| p74-309 | CaMV35S | RosO-TATA-RosO | GUS (see FIGS. 9C, 11) |
| p74-315 | CaMV35S | TATA-RosO | GUS (see FIGS. 9B, 11) |
| p74-316 | CaMV35S | RosO-TATA | GUS (see FIGS. 9A, 11) |
| p74-110 | CaMV35S | TATA-2X RosO | GUS (see FIG. 11) |
| p74-114 | CAMV35S | RosO-TATA-3X | GUS (see FIG. 11) |
| | | RosO | |
| p74-117 | CaMV35S | RosO-TATA-2X | GUS (see FIGS. 9A, 11) |
| | | RosO | |
| p74-118 | CaMV35S | TATA-3X RosO | GUS (see FIGS. 9A, 11) |
| p74-501 | actin 2 | 2X RosO | GUS (see FIG. 9A) |
| p74-502 | actin 2 | TetO | GUS |
| p76-508 | tms2 | 2X RosO | GUS (see FIG. 9D) |

*see 20, FIG. 2
**see 40, FIG. 2
***see 30, FIG. 2

Each of the chimaeric promoters listed in Table 3 was fused to a nucleotide expressing a tag protein, in this case a reporter gene encoding β-glucuronidase (GUS) and introduced into Arabidopsis lines (tag protein lines). When transgenic plant tissues were stained for GUS enzyme activity all of the promoters were determined to be active and functioning in a normal constitutive manner.

Using GUS as a probe, expression of GUS RNA is detected in plants, for example in p74-188 (for construct see FIG. 9A), as indicated in FIG. 12B (GUS parent), or p74-316, p74-118, p74-501 and p74 117 (for constructs see FIG. 9A), as shown in FIG. 13A (GUS) under lanes GUS P1, and GUS P3, GUS P5, and GUS P2, respectively.

Expression of iaaH Gene in Arabidopsis

As an alternate example of a tag protein, the iaaH gene was expressed in Arabidopsis plants under the control of constitutive promoters modified to incorporate the DNA binding sites for either the Tet or Ros repressor proteins (Table 4).

TABLE 4
Conditionally-Lethal Constructs (first nucleotide sequence,
10 see FIG. 2)

| Name | Base Promoter* | Operator** | Lethal Gene*** |
|---|---|---|---|
| p74-311 | actin2 | 2X TetO | iaaH |
| p74-503 | actin2 | 2X RosO | iaaH |
| p76-509 | iaaH | 2X RosO | iaaH |
| p76-510 | iaaH | 2X TetO | iaaH |

*see 20, FIG. 2
**see 40, FIG. 2
***see 30, FIG. 2

Northern blots analysis indicated that the modified actin2 promoters function in a normal constitutive manner to direct the expression of the iaaH gene, for example p74-502 or p74-503 (see FIG. 8, lanes 85 and 86, respectively). The modified iaaH promoters also directed expression of the iaaH gene but at greatly reduced levels relative to the modified actin2 promoter.

Expression of Prokaryotic Repressor Proteins in Arabidopsis

Wild type (wt) or optimized (syn) variants of either the Ros or Tet repressor genes were expressed in Arabidopsis plants under the control of constitutive promoters (Table 5).

TABLE 5
Repressor Constructs (the second nucleotide sequence 50,
see FIG. 2)

| Name | Promoter* | Repressor Gene** |
|---|---|---|
| p74-101 | actin2 | synRos (see FIGS. 9A, 11) |
| p74-107 | CaMV 35S | wtRos (see FIG. 9E) |
| p74-108 | tms 2 | synRos (see FIG. 9F) |
| p74-313 | CaMV 35S | synRo (see FIG. 9A) |
| p76-104 | iaaH | synTet |
| p75-103 | actin2 | synTet |
| p76-102 | CaMV 35S svnTet | |

*see 80, FIG. 2
**see 90, FIG. 2

Western blot analysis indicated that the Ros repressor was expressed effectively in the transgenic lines under the control of modified actin2, CaMV 35S and iaaH promoters (FIG. 10A). Expression of the synthetic Tet protein was detected in plants transformed with construct p75-103 that uses the modified actin2 promoter to direct synTet gene expression (FIG. 10B).

Using ROS as a probe, expression of Ros RNA is detected in plants, for example p74-101 (see FIG. 9A for construct), as indicated in FIG. 12B (ROS parent), or p74-101 as indicated in FIG. 13B, lanes ROS P2 and ROS P3.

### Example 5

Crosses were performed between transgenic A. thaliana and B. napus lines containing repressor constructs and lines containing reporter constructs. To perform the crossing, open flowers were removed from plants of the recipient lines. Fully formed buds of the recipient were gently opened and emasculated to remove all stamens. The stigmas were manually pollinated with pollen from donor lines and pollinated buds were bagged. Once siliques formed, the bags were removed, and mature seeds were collected.

Crossing of Repressor to Conditionally Lethal Lines

Transgenic Arabidopsis lines containing a second nucleotide sequence (50, FIG. 2; repressor constructs) were crossed with lines containing appropriate first nucleotide sequence (10, FIG. 2; conditionally lethal constructs). To perform the crossing, open flowers were removed from plants of the reporter lines. Fully formed buds of plants of the repressor lines were gently opened and emasculated by removing all stamens. The stigmas were then pollinated with pollen from plants of the repressor lines and pollinated buds were tagged and bagged. Once siliques formed, the bags were removed, and mature seeds were collected.

Plants generated from these seeds were then used to determine the level of conditionally lethal gene (iaaH; also known as tms2, encoding the ORF) repression by examination of phenotype following germination on NAM/IAM containing media and spraying plants with NAM/IAM. Levels of iaaH expression in the hybrid lines were compared to those of the original iaaH expressing lines. Plants showing a decrease in iaaH expression levels were further characterized using PCR, Southern and Northern blotting.

The expression of the iaaH gene for use as a positively selectable marker was studied. The system as demonstrated herein, uses two components termed the “lethal” (first nucleotide sequence) and “repressor” constructs (the second nucleotide sequence). The first construct links the iaaH open reading frame (first coding region) to a constitutive promoter that has been altered to incorporate the DNA binding sites (operator sequence) for a transcriptional repressor protein. When introduced into a transgenic plant, the resultant line is sensitized to IAM exposure, or its analogues, as this chemical is converted to IAA causing aberrant cell growth and eventual death of the plant. This line then served as the platform for subsequent transformations. The second construct physically links the coding region of interest (the second coding region) to a third nucleotide coding region encoding a transcriptional repressor protein whose respective DNA binding site resides within the altered iaaH promoter of the first construct. When introduced into the platform line the repressor protein blocks expression of iaaH gene effectively desensitizing these cells to the actions of IAM, allowing such lines to grow in its presence.

Crossing of Lines Expressing Tag Protein with Repressor Lines

Transgenic Arabidopsis or B. napus lines containing repressor constructs (the second nucleotide sequence (50, FIG. 2) are crossed with lines containing appropriate reporter (GUS) constructs (first nucleotide sequences; 10, FIG. 2). To perform the crossing, open flowers are removed from plants of the reporter lines. Fully formed buds of plants of the repressor lines are gently opened and emasculated by removing all stamens. The stigmas are then pollinated with pollen from plants of the repressor lines and pollinated buds are tagged and bagged. Once siliques formed, the bags are removed, and mature seeds are collected. Plants generated from these seeds are then used to determine the level of reporter gene (GUS) repression by GUS staining. Levels of GUS expression in the hybrid lines are compared to those of the original reporter lines. Plants showing a decrease in GUS expression levels are further characterized using PCR, Southern and Northern analysis.

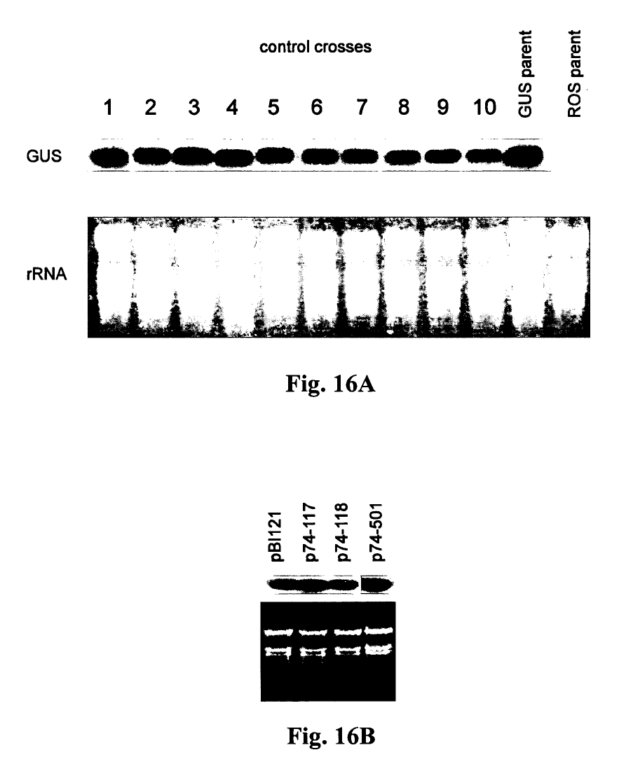

To determine if incorporation of Ros operators into the CaMV35S promoter affected transgene expression, Northern blot analysis was carried out on Arabidopsis lines expressing constructs listed in FIGS. 9 and 11 and lines expressing pBI121. Apart from the natural differences in transgene expression among lines, in general there were no differences in GUS expression that could be attributed to promoter modification. The variability of GUS expression between individual transgenic events did not increase with the modified CaMV35S promoters relative to the unmodified form in pBI121 (FIG. 16), indicating that insertion of the ROS operators in the CaMV35S promoter did not affect its relative ability to initiate transcription.

Repression of GUS Expression by synRos in Arabidopsis

Results of a cross between a transgenic line expressing synthetic Ros, p74-101 and GUS p74-118 (for constructs see FIG. 9A) are presented in FIG. 12.

GUS activity (FIG. 12A) is only observed in plants expressing GUS (termed GUS parent in FIG. 12A, expressing p74-118). The plant expressing ROS (ROS parent, expressing p74-101) exhibited no GUS expression. This result is as expected, since this plant is not transformed with the GUS construct. Of interest, however, is that the plant produced as a result of a cross (Cross in FIG. 12A) between the GUS and ROS parents did not exhibit GUS activity.

Northern analysis (FIG. 12B) demonstrates that GUS expression is consistent with the GUS assay (FIG. 12A), in that only the GUS parent expressed GUS RNA, while no GUS expression was observed in the ROS parent or the progeny arising from a cross between the ROS and GUS parents. Similarly, as expected, no ROS expression was detected in the GUS parent. Ros expression was observed in the ROS parent and in the cross between the ROS and GUS parents.

Southern analysis of the progeny of the cross between the GUS and ROS parents demonstrates that the cross comprised genes encoding both GUS and Ros (FIG. 12C).

These data demonstrate Ros repression of a gene of interest. The progeny of the cross between the ROS and GUS parent lines, comprising both the GUS and Ros gene, expresses the Ros repressor, which binds the operator sequence thereby inhibiting the expression of the gene of interest, in this case GUS. Inhibition of GUS expression was observed at the RNA and protein level, with no enzyme activity was present in the progeny plants.

FIG. 13, shows results of the crosses described in Table 6, between a range of repressor and reporter plants (plants expressing tag protein). Maps of the constructs listed in Table 6 are shown in FIG. 9.

TABLE 6
Crossing of lines expressing reporter lines expressing Tag
Protein (platform plants expressing the first nucleotide
sequence (10)) with Repressor plant lines (expressing the
second nucleotide sequence (50)

| | Constucts | Parental lines |
|---|---|---|
| Crosses | Female × male | Female × male parent |
| Cross1 (C1) | p74-101 × p74-117 | P1GUS × P1ROS |
| Cross2 (C2) | p74-118 × p74-101 | P2ROS × P2GUS |
| Cross3 (C3) | p74-117 × p74-101 | P3GUS × P3ROS |
| Cross4 (C4) | p74-313 × p74-501 | P4GUS × P4ROS |

Northern blot analysis of total RNA (˜4.5 g) isolated from Arabidopsis parental lines including reporter plants expressing a tag protein, in this example GUS, repressor plants (expressing a second nucleotide sequence, 50), and crosses between the parental lines (first nucleotide sequence, 10) as indicated in Table 6 was performed. Results of these analyses are shown in FIGS. 13A-B. The results of GUS expression using GUS as a probe for crosses C1-C4 are shown in FIG. 13A, which also shows the loading of the RNA gel. FIG. 13B shows quantification of the densities of the bands generated in the Northern analysis of FIG. 13A using a GUS probe.

The parental lines expressing Ros, and all of the crosses that were made to Ros exhibited Ros expression (data not shown). No ROS expression is observed in parental lines expressing GUS (reporter constructs) since these lines do not comprise a Ros construct. With reference to FIG. 13A, GUS maximal expression is observed in parental lines expressing a tag protein (also referred to as a reporter construct (GUS P1-P4), however, a range of reduced GUS activity is observed in plants that were crossed (lanes marked C1-C4) with a plants expressing a repressor construct. The range of reduced GUS activity varied with reduction of the maximal GUS activity observed in lines C1D and C1G.

In FIG. 13B, lanes P1&3, P2 GUS, and P4 GUS exhibit GUS expression of the parent expressing the first nucleotide sequence (i.e. p74-316, p74-117, p74-118, p74-117 and p74-501, respectively). These plants exhibit maximum expression of GUS RNA. P1 ROS, P2 ROS, P3 ROS, P4 ROS (comprising p74-101 or p74-313) exhibit background levels of GUS RNA (data not shown), as these plants do not comprise any sequence resulting in GUS expression. Progeny of all crosses between plants expressing the first nucleotide sequence (p74-118, p74-117 and p74-501) and plants expressing the second nucleotide sequence (p74-101 or p74-313) resulted in reduced expression of GUS (the first coding region, 30) by about 30% (for C2B) to about 84% (for C1G).

To show that repression of GUS expression was due to the binding of synRos to the operator sequences in the modified CaMV35S promoters, control crosses were carried out between repressor lines and reporter lines expressing GUS under the control of a CaMV35S promoter without Ros operators, i.e. unaltered (pBI121). No repression of GUS expression was observed in these control crosses (data not shown). This indicates that GUS repression was due to synRos binding to its operator sequences in the re-constructed promoter and affecting GUS expression.

These results show that expression of a tag protein can be controlled using the repressor mediated system as described herein, and that this can be used as basis to select for plants that have been transformed with a nucleotide sequence encoding a coding region of interest.

The present invention provides a selectable marker system that allows the efficient selection of transformed plants utilizing genes that are otherwise benign and confer no adaptive advantage. The benign selectable marker system may facilitate public acceptance of genetically modified organisms by eliminating the issue of antibiotic resistance. Further, the present invention provides a selectable marker system for plant transformation that includes stringent selection of transformed cells, avoids medically relevant antibiotic resistance genes, and provides an inexpensive and effective selection agent that is not-toxic to plant cells.

Repression of GUS Expression by synRos in B. napus

To demonstrate that the ability of synRos to repress gene expression is not restricted to A. thaliana, we tested the synRos repressor system in B. napus. Transgenic B. napus lines were generated that expressed either synRos under the control of the actin2 promoter or the reporter gene GUS under a modified CaMV35S promoter having four Ros operators (p74-114): two flanking the TATA box and two downstream of the transcription initiation site (FIG. 4). This reporter construct was chosen since it incorporated all of the features of the reporter constructs deemed to be functional in A. thaliana.

Agrobacterium-mediated transformation of B. napus was carried out as described in Moloney et al. (1989) with modifications. Seeds were sterilized and then plated on ½ strength hormone-free MS medium (Sigma) with 1% sucrose in 15X60 mm petri dishes. Seeds were then transferred, with the lid removed, into Magenta GA-7 vessels (temperature of 25 degrees C., with 16 h light/8 h dark and a light intensity of 70-80 microE.

Cotyledons were excised from 4-day old seedlings and soaked in BASE solution (4.3 g/L MS (GIBCO BRL), 10 ml 100× B5 Vitamins (0.1 g/L nicotinic acid, 1.0 g/L thiamine-HCl, 0.1 g/L pyridoxine-HCl, 10 g/L m-inositol), 2% sucrose, 1 mg/L 2,4-D, pH 5.8; 1% DMSO and 200 microM acetosyringone added after autoclaving) containing Agrobacterium cells comprising a recombinant plant transformation vector. Most of the BASE solution was removed and the cotyledons were incubated at 28 degrees C. for 2 days in the dark. The dishes containing the cotyledons were then transferred to 4 degrees C. for 3-4 days in the dark. Cotyledons were transferred to plates containing MS B5 selection medium (4.3 g/L MS, 10 ml 100× B5 Vitamins, 3% sucrose, 4 mg/L benzyl adenine (BA) ph 5.8; timentin (300 Fg/ml) and kanamycin (20 Fg/ml) were added after autoclaving) and left at 25 degrees C, 16 h light/8 dark with lighting to 70-100 microE. Shoots were transferred to Magenta GA-7 vessels containing MS B5 selection medium without BA. When shoots were sufficiently big they were transferred to Magenta GA-7 vessels containing rooting medium and upon development of a good root system plantlets were removed from the vessels and transferred to moist potting soil.

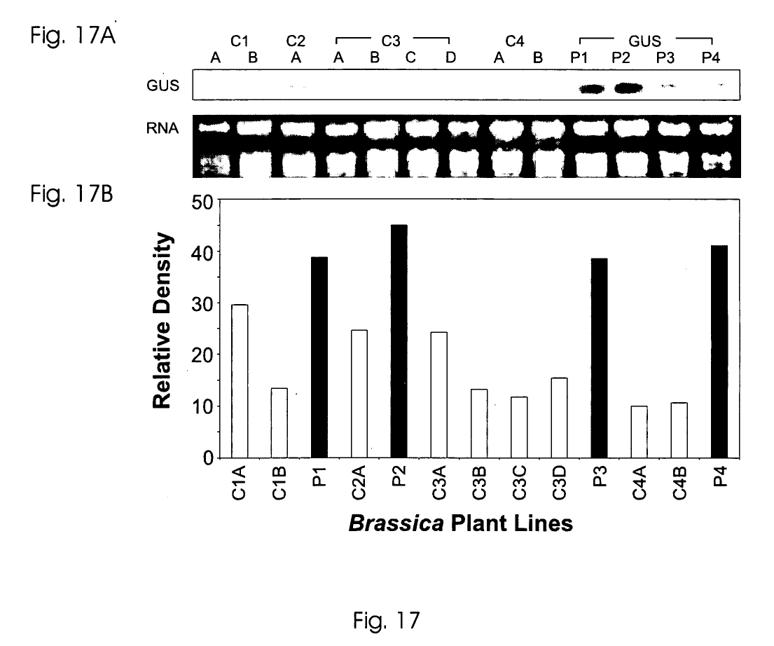

Parental Brassica napus lines separately comprising p74-101 or p74-114 are crossed to produce hybrid lines comprising both p74-101 and p74-114. Crosses performed are as follows: C1 to C4 are p74-114 x p74-101. P1 to P4 are GUS parental lines for crosses C1 to C4. PROS is ROS parent plant for crosses C1 to C4. Levels of GUS expression in the hybrid lines are compared to those of the original parent lines by northern analysis as shown in FIG. 17. FIG. 17 demonstrates that high GUS expression, greater than 100, only occurs in the GUS parental lines P1 and P2, while no GUS expression was observed in the ROS parent PROS (data not shown), and GUS expression is reduced in progeny arising from a cross between the ROS and GUS parents, C1 to C4. Similarly, as expected, no Ros expression was detected in the GUS parental lines, P1 to P4 (data not shown). Ros expression was observed in the ROS parent and in the cross between the ROS and GUS parents (data not shown).

GUS expression was reduced in lines resulting from crosses between the synRos repressor line and GUS reporter lines compared to GUS expression in the parental lines (FIG. 17A). A quantitative assessment of GUS repression by synRos in B. napus indicated that repression ranged from 22% in cross C1A to 66% in cross C5 (FIG. 17B).

These data further demonstrate Ros repression of a gene of interest in Brassicacae. The progeny of the cross between the ROS and GUS parent lines, comprising both the GUS and Ros gene, expresses the Ros repressor, which binds the operator sequence thereby inhibiting the expression of the gene of interest, in this case GUS.

All citations are herein incorporated by reference.

The present invention has been described with regard to preferred embodiments. However, it will be obvious to persons skilled in the art that a number of variations and modifications can be made without departing from the scope of the invention as described herein.

### REFERENCES

An et al., 1996, Plant J., 10: 107-121

Aoyama, T. and Chua, N. H., 1997, Plant J. 2, 397-404

Archdeacon J et al. 2000, FEMS Microbiol Let. 187: 175-178

Arnison P G, Fabijanski S F, Keller W A. (2000) Transgenic plants and methods for production therof. PCT Application WO0037060.

Bittinger et al., (1997) Mol. Plant Microbe Interact. 10: 180-186

Bourgeois S, Pfahl M. (1976) Repressors. Adv Protein Chem. 30:1-99.

Brandstatter, I. and Kieber, J.1, 1998, Plant Cell 10,1009-10 1019

Brightwell et al., (1995) Mol. Plant Microbe Interact. 8: 747-754

Budar F, DeBoeck F, van Montagu M, Hernalsteens J-P. (1986) Introduction and expression of the octopine T-DNA oncogenes in tobacco plants and their progeny. Plant Sci 46: 195-206.

Caddick, M X, et al, 1998, Nature Biotech. 16, 177-180

Carrington et al, 1991, Plant Cell, 3: 953-962

Chou, A. Y., Archdeacon, J. and Kado, C. I. (1998) Agrobacterium transcriptional regulator Ros is a prokaryotic zinc finger protein that regulates the plant oncogene ipt. Proc. Natl. Acad. Sci. USA 95: 5293-5298.

Clough S J, Bent A F. (1998) Floral dip: a simplified method for Agrobacterium-mediated transformation of Arabidopsis thaliana. Plant J. 16:735-743.

Cooley et al. 1991, J. Bacteriol. 173: 2608-2616

Cornejo et al, 1993, Plant Mol Biol. 1993 Nov;23(3):567-81

Depicker A G, Jacobs A M, van Montagu M C. (1988) A negative selection scheme for tobacco protoplast-derived cells expressing the T-DNA gene 2. Plant Cell Reports 7: 63-66.

Deuschle U, Hipskind R A, Bujard H. (1990) RNA polymerase II transcription blocked by Escherichia coli lac repressor. Science 248:480-483.

D'Souza-Ault M. R., 1993, J Bacteriol 175: 3486-3490

Ebinuma H, Sugita K, Matsunaga E, Yamakado M. (1997) Selection of marker-free transgenic plants using the isopentenyl transferase gene. Proc Natl Acad Sci U S A. 94:2117-2121.

Eichholtz D A, Rogers S G, Horsch R B, Klee H J, Hayford M, Hoffmann N L, Braford S B, Fink C, Flick J, O'Connell K M. (1987) Expression of mouse dihydrofolate reductase gene confers methotrexate resistance in transgenic petunia plants. Somat Cell Mol Genet 13:67-76.

Fabijanski S, Arnison P, Robert L, and Schernthaner J. (1999) Methods and genetic compositions to limit out crossing and undesired gene flow in crop plants. PCT WO 00/37660.

Fraley R, Rogers S, Horsch R. (1986) Genetic transformation in higher plants. CRC Crit Rev Plant Sci 4:145.

Gao, M-J., Schäfer, U. A., Parkin, I. A. P., Hegedus, D. D., Lydiate, D. J. and Hannoufa, A. (2003) A novel protein from Brassica napus has a putative KID-domain and responds to low temperature. Plant J. 33: 1073-1086.

Gatz, C.,1997, Ann. Rev. Plant Physiol. Plant Mol. Biol. 48, 89-108

Gatz, C. and Lenk, I. R. P.,1998, Trends Plant Sci. 3, 352-358

Gatz C, Quail P H. (1988) Tn10-encoded tet repressor can regulate an operator-containing plant promoter. Proc Natl Acad Sci U S A. 85:1394-1397.

Gatz C, Kaiser A, Wendenburg R. (1991) Regulation of a modified CaMV 35S promoter by the Tn10-encoded Tet repressor in transgenic tobacco. Mol Gen Genet.227:229-237.

Gatz C, Frohberg C, Wendenburg R. (1992) Stringent repression and homogeneous de-repression by tetracycline of a modified CaMV 35S promoter in intact transgenic tobacco plants. Plant J. 2:397-404.

Geierson and Corey, Plant Molecular Biology, 2d Ed. (1988)

Harlow E and Lane D. (1988). Antibodies: A Laboratory Manual. Cold Spring Harbor Laboratory Press.

Hillen et al., (1984) J.Mol. Biol. 172, 185-201

Holtorf et al, 1995, Plant Mol. Biol. 29: 637-646

Kakimoto, T., 1996, Science 274, 982-985

Kalderon, D., Roberts, B. L., Richardson, W. D. and Smith, A. E. (1984) A short amino acid sequence able to specify nuclear location. Cell 39: 499-509.

Kapila, J., Rycke, R. D., Montagu, M. V. and Angenon, G. (1997) An Agrobacterium-mediated transient gene expression system for intact leaves. Plant Sci. 122: 101-108.

Karlin-Neumann G A, Brusslan J A, Tobin E M. (1991) Phytochrome control of the tms2 gene in transgenic Arabidopsis: a strategy for selecting mutants in the signal transduction pathway. Plant Cell. 3:573-582.

Keller M et al., (1995) Mol. Plant Microbe Interact. 8: 267-277

Koziel M G, Adams T L, Hazlet M A, Damm D, Miller J, Dahlbeck D, Jayne S, Staskawicz B J. (1984) A cauliflower mosaic virus promoter directs expression of anamycin resistance in morphogenic transformed plant cells. J Mol Appl Genet 2:549-562.

Kunkel T, Niu Q W, Chan Y S, Chua N H. (1999) Inducible isopentenyl transferase as a high-efficiency marker for plant transformation. Nat Biotechnol. 17:916-919.

Lin J-J, Ma J, Garcia-Assad N, Kuo J. (1996) Hygromycin B as an efficient antiobiotic for the selection of transgenic plants. Focus 18:47-49.

Mandel et al, 1995 Plant Mol. Biol. 29: 995-1004

Margraff et al.,1980. Experimentia 36:846

Miki and Iyer, Fundamentals of Gene Transfer in Plants. In Plant Metabolism, 2d Ed. D T. Dennis, D H Turpin, D D Lefebrve, D B Layzell (eds), Addison Wesly, Langmans Ltd. London, pp. 561-579 (1997)

Moloney, M., Walker, J. and Sharma, K. (1989) High efficiency transformation of Brassica napus using Agrobacterium vectors. Plant Cell Rep. 8: 238-242.

Moore I, Galweiler L, GRosskopf D, Schell J, Palme K. (1998) A transcription activation system for regulated gene expression in transgenic plants. Proc Natl Acad Sci U S A. 95:376-381.

Murray E, Lotzer J, and Eberle M. (1989) Codon usage in plant genes. Nucl. Acids. Res. 1989 17: 477-498.

Odell et al., 1985, Nature, 313: 810-812

Owens et al., 1973. Weed Science 21:63-66

Paine et al., (1975) Nature 254:109-112

Perez P, Tiraby G, Kallerhoff J, Perret J. (1989) leomycin resistance as a dominant selectable marker for plant cell transformation. Plant Mol Biol 13:365-373.

Perl A, Shaul O, Galili G. (1992) Regulation of lysine synthesis in transgenic potato plants expressing a bacterial dihydrodipicolinate synthase in their chloroplasts. Plant Mol Biol. 19:815-23.

Petolino J F, Young S, Hopkins N, Sukhapinda K, Woosley A, Hayes C, Pelcher L. (2000) Expression of murine adenosine deaminase (ADA) in transgenic maize. Transgenic Res. 9:1-9.

Privalle L S, Wright M, Reed J, Hansen G, Dawson J, Dunder E M, Chang Y-F, Powell M L, Meghi M. (2000) Phosphomannose isomerase—a novel system for plant selection. Proceedings of the 6th International Symposium on The Biosafety of Genetically Modified Organisms. Saskatoon, SK.

Rizzo P, Di Resta I, Powers A, Ratner H, Carbone M. (1999) Unique strains of SV40 in commercial poliovaccines from 1955 not readily identifiable with current testing for SV40 infection. Cancer Res. 59: 6103-6108.

Robbins et al., 1991, Cell, 64: 615-623

Salter, M. G., et al, 1998, Plant Journal 16, 127-132

Sardana R, Dukiandjiev S, Giband M, Cheng X, Cowan K, Sauder C, Altosaar I. (1996) Construction and rapid testing of synthetic and modified toxin gene sequences CryIA (b & c) by expression in maize endosperm culture. Plant Cell Reports 15: 677-681.

Sonnewald U, Ebneth M. (2000) 2-deoxyglucose-6-phosphate (2-DOG-6-P) phosphatase DNA sequences as selection markers in plants. W09845456C1.

Ulmasov, T., et al., 1997, Plant Cell 9, 1963- 1971

van der Krol and Chua, 1991, Plant Cell, 3: 667-675

Varagona, M. J., Schmidt, R. J. and Raikhel, N. V. (1991) Monocot regulatory protein Opaque-2 is localized in the nucleus of maize endosperm and transformed tobacco plants. Plant Cell 3: 105-113.

Varagona et al., 1992, Plant Cell, 4: 1213-1227

Weissbach and Weissbach, Methods for Plant Molecular Biology, Academy Press, New York VIII, pp. 421-463 (1988)

Xu et. al.,1994, Plant Physiol. 106: 459-467

Yanovsky et al., 1990, Nature, 346: 35-39

Ye G N, Hajdukiewicz P T, Broyles D, Rodriguez D, Xu C W, Nehra N, Staub J M. (2001) Plastid-expressed 5-enolpyruvylshikimate-3-phosphate synthase genes provide high level glyphosate tolerance in tobacco. Plant J. 25:261-70.

Zhang et al, 1991, Plant Cell, 3: 1155-1165

We claim:

1. A method of selecting for a plant or portion thereof that comprises a coding region of interest, the method comprising,
i) providing a platform plant, or portion thereof comprising a first nucleotide sequence comprising,
a first regulatory region in operative association with a first coding region, and an operator sequence, the first coding region encoding a tag protein;
ii) introducing a second nucleotide sequence into the platform plant, or portion thereof to produce a dual transgenic plant, the second nucleotide sequence comprising,
a second regulatory region, in operative association with a second coding region, and a third regulatory region in operative association with a third coding region , the second coding region comprising a coding region of interest, the third coding region encoding a repressor capable of binding to the operator sequence thereby inhibiting expression of the first coding region, and;
iv) selecting for the dual transgenic plant by identifying plants, or portions thereof deficient in the tag protein, expression of the first coding region, or an identifiable genotype or phenotype of the dual transgenic plant associated therewith.

2. The method of claim 1 wherein the plant or portion thereof comprises plant cells, tissue, or the entire plant.

3. The method of claim 1, wherein the plant, or portion thereof is selected from the group consisting of canola, Brassica spp., maize, tobacco, alfalfa, rice, soybean, pea, wheat, barley, sunflower, potato, tomato, and cotton.

4. The method of claim 1, wherein the first coding region is selected from the group consisting of a reporter protein, an enzyme, an antibody and a conditionally lethal coding region.

5. The method of claim 4, wherein the conditionally lethal coding region is selected from the group consisting of indole acetamide hydrolase, methoxinine dehydrogenase, rhizobitoxine synthase, and L-N-acetyl-phosphinothricin deacylase.

6. The method of claim 1, wherein the repressor and the operator sequence are selected from the group consisting of
a) Ros repressor and Ros operator sequence;
b) Tet repressor and Tet operator sequence;
c) Sin3 repressor and Sin 3 operator sequence; and
d) UMe6 repressor and UMe6 operator sequence.

7. The method of claim 6 wherein the repressor and the operator sequence are the Ros repressor and Ros operator sequence.

8. The method of claim 6 wherein the repressor and the operator sequence are the Tet repressor and Tet operator sequence.

9. The method of claim 1 wherein the coding region of interest encodes a pharmaceutically active protein.

10. The method of claim 9, wherein the pharmaceutically active protein is selected from the group consisting of growth factors, growth regulators, antibodies, antigens, interleukins, insulin, G-CSF, GM-CSF, HPG-CSF, M-CSF, interferons, blood clotting factors, transcriptional protein or nutraceutical protein.

11. A method of selecting for a transgenic plant or portion thereof comprising a coding region of interest, the method comprising,
i) transforming the plant, or portion thereof, with a first nucleotide sequence to produce a transformed plant, the first nucleotide sequence comprising a first regulatory region in operative association with a first coding region, and an operator sequence, the first coding region encoding a conditionally lethal protein;
ii) screening for the transformed plant;
iii) introducing a second nucleotide sequence into the transformed plant or portion thereof to produce a dual transgenic plant, the second nucleotide sequence comprising a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region, and;
iv) selecting for the dual transgenic plant by exposing the transformed plant and the dual transformed plant to conditions that permit the conditionally lethal coding region to become conditionally lethal, thereby reducing the growth, development or killing the transformed plant.

12. The method of claim 11, wherein the first regulatory region, secondary regulatory region and third regulatory region are constitutively active in the plant cells.

13. The method of claim 11, wherein the first regulatory region and secondary regulatory region are constitutively active and the third regulatory region is developmentally regulated or inducible.

14. A method of selecting for a transgenic plant or portion thereof comprising a coding region of interest, the method comprising,
i) introducing a second nucleotide sequence into a transformed plant, or portion thereof that comprises a first nucleotide sequence to produce a dual transgenic plant, the first nucleotide sequence comprising a first regulatory region in operative association with a first coding region, and an operator sequence, the first coding region encoding a tag protein,
and wherein the second nucleotide sequence comprises a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region, and;
ii) selecting for the dual transgenic plant.

15. A method of selecting for a transgenic plant or portion thereof comprising a coding region of interest, the method comprising,
i) transforming the plant, or portion thereof, with a first nucleotide sequence to produce a transformed plant, the first nucleotide sequence comprising a first regulatory region in operative association with a first coding region, and an operator sequence, the first coding region encoding a tag protein;
ii) screening for the transformed plant;
iii) introducing a second nucleotide sequence into the transformed plant or portion thereof to produce a dual transgenic plant, the second nucleotide sequence comprising a second regulatory region in operative association with a second coding region encoding a fusion-protein, the fusion protein comprising a protein of interest fused to a repressor capable of binding to the operator sequence of the first coding region thereby inhibiting expression of the first coding region, and;
iv) selecting for the dual transgenic plant.

16. The method of claim 15, wherein the fusion protein additionally comprises at least one of: a) a linker region linking the repressor to the protein of interest and b) an affinity tag.

17. The method of claim 16, wherein the linker region is enzymatically cleavable.

18. The method of claim 15, wherein the fusion protein has a molecular mass below about 100 kDa.

19. The method of claim 15, wherein the fusion protein has a molecular mass below about 65 kDa.

20. A plant cell, tissue, seed or plant comprising,
i) a first nucleotide sequence comprising a first regulatory region in operative association with a first coding region and an operator sequence, the first coding region encoding a tag protein, and;
ii) a second nucleotide sequence comprising a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region.

21. The plant cell, tissue, seed or plant of claim 20, wherein the first coding region is selected from the group consisting of a reporter protein, an enzyme, an antibody and a conditionally lethal coding region.

22. A plant cell, tissue, seed or plant comprising,
i) a first nucleotide sequence comprising a first regulatory region in operative association with a first coding region and an operator sequence, the first coding region encoding a tag protein, and;
ii) a second nucleotide sequence comprising a second regulatory region in operative association with a second coding region, the second coding region encoding a fusion-protein, the fusion-protein comprising a protein of interest fused to a repressor capable of binding to the operator sequence thereby inhibiting expression of the first coding region.

23. A plant cell, tissue, seed or plant comprising, a first nucleotide sequence comprising a first regulatory region in operative association with a first coding region and an operator sequence, the first coding region encoding a tag protein.

24. A plant cell, tissue, seed or plant comprising, a second nucleotide sequence comprising a second regulatory region in operative association with a second coding region, and a third regulatory region in operative association with a third coding region, the second coding region comprising a coding region of interest, the third coding region encoding a repressor capable of binding to an operator sequence.

25. A construct comprising, a first nucleotide sequence comprising a first regulatory region in operative association with a first coding region and an operator sequence, the first coding region encoding a tag protein.

26. A construct comprising a second nucleotide sequence comprising a second regulatory region in operative association with a second coding region, and a third regulatory region in operative association with a third coding region, the second coding region comprising a coding region of interest, the third coding region encoding a repressor capable of binding to an operator sequence.

27. A pair of constructs comprising,
i) a first nucleotide sequence comprising a first regulatory region in operative association with a first coding region and an operator sequence, the first coding region encoding a tag protein, and;
ii) a second nucleotide sequence comprising a second regulatory region in operative association with a second coding region, and a third regulatory region in operative association with a third coding region, the second coding region comprising a coding region of interest, the third coding region encoding a repressor capable of binding to the operator sequence thereby inhibiting expression of the first coding region.

28. A pair of constructs comprising,
i) a first nucleotide sequence comprising a first regulatory region in operative association with a first coding region and an operator sequence, the first coding region encoding a tag protein, and;
ii) a second nucleotide sequence comprising a second regulatory region in operative association with a second coding region, the second coding region encoding a fusion-protein, the fusion-protein comprising a protein of interest fused to a repressor capable of binding to the operator sequence thereby inhibiting expression of the first coding region.

29. A method of selecting for a plant or portion thereof that comprises a coding region of interest, the method comprising,
i) transforming a plant, or portion thereof with a first nucleotide sequence to produce a transformed plant, the first nucleotide sequence comprising,
a first regulatory region in operative association with a first coding region, and an operator sequence, the first coding region encoding a tag protein;
ii) introducing a second nucleotide sequence into the transformed plant, or portion thereof to produce a dual transgenic plant, the second nucleotide sequence comprising,
a second regulatory region, in operative association with a second coding region, and a third regulatory region in operative association with a third coding region , the second coding region comprising a coding region of interest, the third coding region encoding a repressor capable of binding to the operator sequence thereby inhibiting expression of the first coding region, and;
iv) selecting for the dual transgenic plant by identifying plants, or portions thereof deficient in the tag protein, expression of the first coding region, or an identifiable genotype or phenotype of the dual transgenic plant associated therewith.


SEQUENCE LISTING (61 sequences)

SEQ ID NO: 1 - DNA, 472 bp, artificial
  Synthetic Ros optimized for plant expression
gcggatccccgggtatgactgagactgcttacggtaacgctcaggatcttcttgttgagcttactgctgatatcgttgctgcttacgtttctaaccacgttgttcctgttactgagcttcctggacttatctctgatgttcatactgcactttctggaacatctgctcctgcttctgttgctgttaacgttgagaagcagaagcctgctgtttctgttcgtaagtctgttcaggatgatcatatcgtttgtttggagtgtggtggttctttcaagtctctcaagcgtcaccttactactcatcactctatgactccagaggagtatagagagaagtgggatcttcctgttgattaccctatggttgctcctgcttacgctgaggctcgttctcgtctcgctaaggagatgggtctcggtcagcgtcgtaaggctaaccgtccaaaaaagaagcgtaaggtctgagagctcgc

SEQ ID NO: 2 - DNA, 678 bp, artificial
  Synthetic Tet optimized for plant expression
ggtaccgagaaaatgtctagattagataaaagtaaagtgattaacagcgcattagagctgcttaatgaggtcggaatcgagggcttaacgacccgtaaactcgcgcagaagctaggagtagagcagcctacgttgtactggcatgttaagaacaagcgggctttgctcgacgccctcgcgattgagatgttagacaggcaccatactcacttctgccctctcgaaggggagagctggcaagatttcctccgtaacaacgctaagtccttcagatgtgctctcctatcccatcgcgacggagcaaaagttcatctgggtacacggcctacagagaaacagtatgagactctcgaaaatcaactggcctttctgtgccaacagggtttctcactagagaatgcgctttacgcactctcagctgtggggcattttactcttggttgcgttttggaggatcaagagcatcaagtcgctaaggaagagagggaaacacctactactgatagtatgccgccacttcttcgacaagccatcgaactttttgatcaccagggtgcagagccagccttcttgttcggccttgaattgatcatatgcggattggaaaagcagcttaaatgtgaatcggggtctcttaagccaaaaaagaagcgtaaggtctgacttaagtgaatcgat

SEQ ID NO: 3 - protein, 149 aa, Artificial
  Synthetic Ros
MTETAYGNAQDLLVELTADIVAAYVSNHVVPVTELPGLISDVHTALSGTSAPASVAVNVEKQKPAVSVRKSVQDDHIVCLECGGSFKSLKRHLTTHHSMTPEEYREKWDLPVDYPMVAPAYAEARSRLAKEMGLGQRRKANRPKKKRKV

SEQ ID NO: 4 - protein, 216 aa, Artificial
  Synthetic Tet
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGSLKPKKKRKV

SEQ ID NO: 5 - DNA, 24 bp, Artificial
  Actin2 promoter sense primer
aagcttatgtatgcaagagtcagc

SEQ ID NO: 6 - DNA, 24 bp, Artificial
  Actin2 promoter anti-sense primer
ttgactagtatcagcctcagccat

SEQ ID NO: 7 - DNA, 27 bp, Artificial
  Ros sense primer
gcggatccgatgacggaaactgcatac

SEQ ID NO: 8 - DNA, 25 bp, Artificial
  Ros anti-sense primer
gcaagcttcaacggttcgccttgcg

SEQ ID NO: 9 - DNA, 36 bp, Artificial
  iaaH sense primer
tgcggatgcataagcttgctgacattgctagaaaag

SEQ ID NO: 10 - DNA, 26 bp, Artificial
  iaaH anti-sense primer
cggggatcctttcagggccatttcag

SEQ ID NO: 11 - DNA, 43 bp, Artificial
  Tet-FI primer
gatcactctatcagtgatagagtgaactctatcagtgatagag

SEQ ID NO: 12 - DNA, 41 bp, Artificial
  Tet-RI primer
cgctctatcactgatagagttcactctatcactgatagagt

SEQ ID NO: 13 - DNA, 26 bp, Artificial
  iaaH ORF sense primer
gctctagaatggtgcccattacctcg

SEQ ID NO: 14 - DNA, 26 bp, Artificial
  iaaH ORF anti-sense primer
gcgagctcawatggcttyttcyaatg

SEQ ID NO: 15 - DNA, 59 bp, Artificial
  Ros-OP1
gatcctatatttcaattttattgtaatatagctatatttcaattttattgtaatataat

SEQ ID NO: 16 - DNA, 57 bp, Artificial
  Ros-OP2
cgattatattacaataaaattgaaatatagctatattacaataaaattgaaatatag

SEQ ID NO: 17 - DNA, 25 bp, Agrobacterium tumefaciens
tatatttcaattttattgtaatata

SEQ ID NO: 18 - DNA, 27 bp, Agrobacterium tumefaciens
tataattaaaatattaactgtcgcatt

SEQ ID NO: 19 - DNA, 429 bp, Agrobacterium tumefaciens
atgacggaaactgcatacggtaacgcccaggatctgctggtcgaactgacggcggatattgtggctgcctatgttagcaaccacgtcgttccggtaactgagcttcccggccttatttcggatgttcatacggcactcagcggaacatcggcaccggcatcggtggcggtcaatgttgaaaagcagaagcctgctgtgtcggttcgcaagtcggttcaggacgatcatatcgtctgtttggaatgtggtggctcgttcaagtcgctcaaacgccacctgacgacgcatcacagcatgacgccggaagaatatcgcgaaaaatgggatctgccggtcgattatccgatggttgctcccgcctatgccgaagcccgttcgcggctcgccaaggaaatgggtctcggtcagcgccgcaaggcgaaccgttga

SEQ ID NO: 20 - DNA, 624 bp, escherichia coli
atgtctagattagataaaagtaaagtgattaacagcgcattagagctgcttaatgaggtcggaatcgaaggcctaacaacccgtaaacttgcgcagaagctcggggtagagcagcctacattgtattggcatgtaaaaaataagcgggccctgctcgacgcgttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaa

SEQ ID NO: 21 - protein, 142 aa, Agrobacterium tumefaciens
MTETAYGNAQDLLVELTADIVAAYVSNHVVPVTELPGLISDVHTALSGTSAPASVAVNVEKQKPAVSVRKSVQDDHIVCLECGGSFKSLKRHLTTHHSMTPEEYREKWDLPVDYPMVAPAYAEARSRLAKEMGLGQRRKANR

SEQ ID NO: 22 - protein, 207 aa, Escherichia coli
MSRLDKSKVINSALELLNEVGIEGLTTRKLAQKLGVEQPTLYWHVKNKRALLDALAIEMLDRHHTHFCPLEGESWQDFLRNNAKSFRCALLSHRDGAKVHLGTRPTEKQYETLENQLAFLCQQGFSLENALYALSAVGHFTLGCVLEDQEHQVAKEERETPTTDSMPPLLRQAIELFDHQGAEPAFLFGLELIICGLEKQLKCESGS

SEQ ID NO: 23 - DNA, 10 bp, Artificial
  Consensus Ros operator sequence
watdhwkmar

SEQ ID NO: 24 - protein, 7 aa, SV40
PKKKRKV

SEQ ID NO: 25 - DNA, 109 bp, Artificial
  Ros-OPDS
atctccactgacgtaagggatgacgcacaatcccactatccttcgcaagacccttcctctatataatatatttcaattttattgtaatataacacgggggactctagag

SEQ ID NO: 26 - DNA, 113 bp, Artificial
  Ros-OPDA
gatcctctagagtcccccgtgttatattacaataaaattgaaatatattatatagaggaagggtcttgcgaaggatagtgggattgtgcgtcatcccttacgtcagtggagat

SEQ ID NO: 27 - DNA, 138 bp, Artificial
  p74-315 sequence from EcoRV to ATG of GUS
gatatctccactgacgtaagggatgacgcacaatcccactatccttcgcaagacccttcctctatataatatatttcaattttattgtaatataacacgggggactctagaggatccccgggtggtcagtcccttatg

SEQ ID NO: 28 - DNA, 107 bp, Artificial
  Ros-OPUS
atctccactgacgtaagggatgacgcacaatctatatttcaattttattgtaatatactatataaggaagttcatttcatttggagagaacacgggggactctagag

SEQ ID NO: 29 - DNA, 111 bp, Artificial
  Ros-OPUA
gatcctctagagtcccccgtgttctctccaaatgaaatgaacttccttatatagtatattacaataaaattgaaatatagattgtgcgtcatcccttacgtcagtggagat

SEQ ID NO: 30 - DNA, 136 bp, Artificial
  p74-316 sequence from EcoRV to ATG of GUS
gatatctccactgacgtaagggatgacgcacaatctatatttcaattttattgtaatatactatataaggaagttcatttcatttggagagaacacgggggactctagaggatccccgggtggtcagtcccttatg

SEQ ID NO: 31 - DNA, 108 bp, Artificial
  Ros-OPPS
atctccactgacgtaagggatgacgcacaatctatatttcaattttattgtaatatactatataatatatttcaattttattgtaatataacacgggggactctagag

SEQ ID NO: 32 - DNA, 112 bp, Artificial
  Ros-OPPA
gatcctctagagtcccccgtgttatattacaataaaattgaaatatattatatagtatattacaataaaattgaaatatagattgtgcgtcatcccttacgtcagtggagat

SEQ ID NO: 33 - DNA, 137 bp, Artificial
  p74-309sequence from EcoRV to ATG of GUS
gatatctccactgacgtaagggatgacgcacaatctatatttcaattttattgtaatatactatataatatatttcaattttattgtaatataacacgggggactctagaggatccccgggtggtcagtcccttatg

SEQ ID NO: 34 - DNA, 237 bp, Artificial
  p74-118 sequence from EcoRV to ATG of GUS
gatatctccactgacgtaagggatgacgcacaatcccactatccttcgcaagacccttcctctatataatatatttcaattttattgtaatataacacgggggactctagaggatcctatatttcaattttattgtaatatagctatatttcaattttattgtaatataatcgatttcgaacccggggtaccgaattcctcgagtctagaggatccccgggtggtcagtcccttatg

SEQ ID NO: 35 - DNA, 235 bp, Artificial
  p 74-117 sequence from EcoRV to ATG of GUS
gatatctccactgacgtaagggatgacgcacaatctatatttcaattttattgtaatatactatataaggaagttcatttcatttggagagaacacgggggactctagaggatcctatatttcaattttattgtaatatagctatatttcaattttattgtaatataatcgatttcgaacccggggtaccgaattcctcgagtctagaggatccccgggtggtcagtcccttatg

SEQ ID NO: 36 - protein, 16 aa, Arabidopsis
RIENTTNRQVTFCKRR

SEQ ID NO: 37 - protein, 18 aa, Tobacco
RRLAQNREAARKSRIRKK

SEQ ID NO: 38 - protein, 20 aa, Tobacco
KKRARLVNRESAQLSRQRKK

SEQ ID NO: 39 - protein, 18 aa, Maize
RKRKESNRESARRSRYRK

SEQ ID NO: 40 - protein, 45 aa, Potyvirus
  MISC_FEATURE (11)..(42): where Xaa is any amino acid
KKNQKHKLKMXXXXXXXXXXXXXXXXXXXXXXXXXXXXXXXXKRK

SEQ ID NO: 41 - protein, 17 aa, Xenopus
KRPAATKKAGQAKKKKI

SEQ ID NO: 42 - protein, 17 aa, Xenopus
KRIAPDSASKVPRKKTR

SEQ ID NO: 43 - protein, 17 aa, Xenopus
KRKTEEESPLKDKDAKK

SEQ ID NO: 44 - protein, 17 aa, Rat
RKCLQAGMNLEARKTKK

SEQ ID NO: 45 - protein, 17 aa, Human
RKCLQAGMNLEARKTKK

SEQ ID NO: 46 - protein, 17 aa, Human
RKCLQAGMNLEARKTKK

SEQ ID NO: 47 - protein, 17 aa, Chicken
RKCCQAGMVLGGRKFKK

SEQ ID NO: 48 - protein, 17 aa, Human
RKCYEAGMTLGARKIKK

SEQ ID NO: 49 - protein, 17 aa, Chicken
RRCFEVRVCACPGRDRK

SEQ ID NO: 50 - DNA, 236 bp, Artificial
  p74-114 sequence from EcoRV to ATG of GUS
gatatctccactgacgtaagggatgacgcacaatctatatttcaattttattgtaatatactatataatatatttcaattttattgtaatataacacgggggactctagaggatcctatatttcaattttattgtaatatagctatatttcaattttattgtaatataatcgatttcgaacccggggtaccgaattcctcgagtctagaggatccccgggtggtcagtcccttatg

SEQ ID NO: 51 - DNA, 33 bp, Artificial
  synRos forward primer
gcggatccatgactgagactgcttacggtaacg

SEQ ID NO: 52 - DNA, 29 bp, Artificial
  synRos reverse primer
gcgagctcgaccttacgcttcttttttgg

SEQ ID NO: 53 - DNA, 26 bp, Artificial
  wtRos forward primer
cgggatccatgacggaaactgcatac

SEQ ID NO: 54 - DNA, 24 bp, Artificial
  wtRos reverse primer
gcgagctcacggttcgccttgcgg

SEQ ID NO: 55 - DNA, 108 bp, Artificial; identical to SEQ ID NO: 31
  Ros oligonucleotide for Southwestern

SEQ ID NO: 56 - DNA, 43 bp, Artificial; identical to SEQ ID NO: 11
  Tet oligonucleotide for Southwestern

SEQ ID NO: 57 - DNA, 10 bp, Agrobacterium tumefaciens
tatatttcaa

SEQ ID NO: 58 - DNA, 10 bp, Agrobacterium tumefaciens
tatattacaa

SEQ ID NO: 59 - DNA, 10 bp, Agrobacterium tumefaciens
tataattaaa

SEQ ID NO: 60 - DNA, 10 bp, Agrobacterium tumefaciens
aatgcgacag

SEQ ID NO: 61 - DNA, 10 bp, Artificial
  Ros operator sequence (1)
tatahttcaa